SEQUENCE LISTING (13 sequences)

SEQ ID NO: 1 - DNA, 30 bp, Artificial Sequence
  Description of Artificial Sequence Synthetic primer
aaacacctctaggctataaggcaacagagc

SEQ ID NO: 2 - DNA, 24 bp, Artificial Sequence
  Description of Artificial Sequence Synthetic primer
aagtaaacttccacaaccgcaagc

SEQ ID NO: 3 - DNA, 22 bp, Artificial Sequence
  Description of Artificial Sequence Synthetic primer
ggagctagaacgattcgcagtt

SEQ ID NO: 4 - DNA, 24 bp, Artificial Sequence
  Description of Artificial Sequence Synthetic primer
gttgtagctgtcccagtatttgtc

SEQ ID NO: 5 - DNA, 30 bp, Artificial Sequence
  Description of Artificial Sequence Synthetic probe
acagccttctgatgtttctaacaggccagg

SEQ ID NO: 6 - DNA, 19 bp, Artificial Sequence
  Description of Artificial Sequence Synthetic primer
tcacccacactgtgcccat

SEQ ID NO: 7 - DNA, 18 bp, Artificial Sequence
  Description of Artificial Sequence Synthetic primer
agccaggtccagacgcag

SEQ ID NO: 8 - DNA, 26 bp, Artificial Sequence
  Description of Artificial Sequence Synthetic probe
tacgagggctatgctctccctcacgc

SEQ ID NO: 9 - DNA, 356 bp, Homo sapiens
aacatatttatcgggcatttctgagcaactaactcatgcaggactctcaaacactaacctatagccttttctatgtatctacttgtgtagaaaccaagcgtggggactgagaaggcaatagcaggagcattctgactctcactgcctttggctaggtccctccctcatcacagctcagcatagtccgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaa

SEQ ID NO: 10 - DNA, 350 bp, Galago sp.
gacacattcatcaggcatatctgagcaattaaaaacatgtagtaccctcaaactcagacccttccatttatctacacatgtagaaaccaaagatggggactgagaaggtagcagtgagagagttctggctctcactgtctcacagtggttcctcctgagccacagccctgtgtaatcctaacacttatctttatccacagttcctgaaccagcccagctatcactaactcaagtccccagcagaaaaatactgggtctgaatgaccctgttgattaaaactaaaacaaaacaaaacaaaacaaggtggcaaacctcccacaaaacataactaaataagcaaaaagaaaaa

SEQ ID NO: 11 - DNA, 351 bp, Unknown
  Description of Unknown Rabbit polynucleotide
aatgtattttcagaggatttctgagcaattaaccacacaggacccttaaatcctaacccacaccactttgccttcagtatgctacctgtgtgggaaccagggatggggacacagaagtcagaagcactccaggtttctgttgttagccagggccctcccttaccatagcttcgtgtcacactgagccttatctatacccacagtttctgaatccacctggctatcaccagctcagtccttaggagaagggaaaacaaacactgagtttgtctctgcttataaaaacaaagcaaaataaaattaaatttcaatcccatcccaaacaaatgaataactaagcaaaaacaaaaa

SEQ ID NO: 12 - DNA, 343 bp, Unknown
  Description of Unknown Goat polynucleotide
ggcatatttactgggcatttctgaacagttaaccatgcaagacccccaggcacaaacccataatctctttcacttatctacttgtatggaaacaaaagacagagtctttgaaagcagtaagagtagcaccctggctttcattgtctttagctggtcctctccttgtgtactcctgagcccttatctatgttcacagtttctgacccatccagctatcttcagcccaagaccccagaaggagaaagacactggatcggcttccctttcttaattgaaaacaaaataaattaaatgccatccccacaagtcccagaaaacttgtgaacacatagaaggagaagga

SEQ ID NO: 13 - DNA, 342 bp, Mus sp.
aacgtatttatcaggctttcctaagccagatcctcaaacactctcccataacccatacctcttatctactcatgaaggagcaatgatgggacaaagaaggcaggaccccaaacacgctggcttccctcaatctcacggaggccccaaccttgactcagctctgtgcagctctggctcttatctgtcaccacagtctgtgactccacccagctgacaccaccccaagtcccagaagaagaaaaacaccagttctttctacttctacttatttaaataaaataaaataaaagaaagccttacatccctggacagataaacaagaataaaaagaaagaagaaaga

(19) United States
(12) Patent Application Publication
(10) Pub. No.: US 2009/0156534 A1
(43) Pub. Date: Jun. 18, 2009

(54) GLOBIN LENTIVIRAL VECTORS FOR TREATMENT OF DISEASE

(75) Inventors: Leszek Lisowski, Foster City, CA (US); Michel Sadelain, New York, NY (US)

Correspondence Address: EDWARDS ANGELL PALMER & DODGE, LLP, P.O. BOX 55874, BOSTON, MA, 02205, US

(73) Assignee: MEMORIAL SLOAN-KETTERING CANCER CENTER, New York, NY (US)

(21) Appl. No.: 12/209,913

(22) Filed: Sep. 12, 2008

Related U.S. Application Data
(60) Provisional application No. 60/993,805, filed on Sep. 13, 2007.
(60) Provisional application No. 61/049,737, filed on May 1, 2008.

(51) Int. Cl. A61K 31/7088; C12N 15/74; C12P 21/00; A61P 7/06

(52) U.S. Cl. 514/44; 435/320.1; 435/69.1

(57) ABSTRACT

The invention provides compositions and methods for the treatment or prevention of disease, including, for example, β-thalassemia, anemias (e.g., sickle cell anemia) and other hemoglobinopathologies.

### CROSS-REFERENCE TO RELATED APPLICATIONS

This application is claims priority to U.S. provisional patent application Ser. No. 61/049,737, filed May 1, 2008 and to U.S. provisional patent application Ser. No. 60/993,805, filed Sep. 13, 2007, the entire disclosures of which are hereby incorporated herein by reference. This application may be related to International Patent Publication Nos.: WO2003/002155 and WO2007/044627, the disclosures of which are hereby incorporated herein in their entireties by reference.

### STATEMENT OF RIGHTS TO INVENTIONS MADE UNDER FEDERALLY SPONSORED RESEARCH

This work was supported by the following grant from the National Institutes of Health, Grant No: RO1 HL-57612. The government has certain rights in the invention.

### BACKGROUND OF THE INVENTION

The β-thalassemias are inherited autosomal recessive anemias caused by mutations that diminish or abolish expression of the β-globin gene. The only current means to cure the disease is allogeneic bone marrow transplantation (BMT). In the absence of a histocompatible donor, however, the genetic correction of autologous hematopoietic stem cells (HSCs) represents a highly attractive alternative treatment because of its curative potential without the risk of graft-versus-host disease.

### SUMMARY OF THE INVENTION

The invention provides compositions and methods for the treatment or prevention of disorders including thalassemia, anemia (e.g., sickle cell anemia) or another hemoglobinopathology.

In one aspect, the invention provides a recombinant vector comprising a nucleotide sequence encoding a heterologous protein; a nucleotide sequence encoding an enhancer of a β-globin locus control region (LCR) which consists of operably joined Dnase1 hypersensitive site (HS) spanning-fragments; and a nucleotide sequence encoding an HS1 fragment consisting of about nucleotides 20900 to 21973 of the human β-globin LCR sequence of GenBank Accession No. NG_{—}000007.3 (NG7) or the corresponding sequences from a mammalian β-globin LCR; the vector providing expression of the protein when introduced into a mammal in vivo.

In one embodiment, the vector further comprises a β-globin promoter. In another embodiment, the HS spanning-fragments span HS2, HS3 and HS4. In a specific embodiment, the HS1 fragment lies between the β-globin promoter and an HS-spanning fragment, and wherein the HS-spanning fragment is an HS2-spanning fragment. In another specific embodiment, the β-globin promoter is ≧ about 265 bp in length. In another specific embodiment, the β-globin promoter is about 615 bp in length.

In another embodiment, the HS spanning-fragments span HS2 and HS3. In still another embodiment, the HS spanning-fragments consist essentially of an HS2-spanning nucleotide fragment extending between BstXI and SnaBI restriction sites of said LCR, an HS3-spanning nucleotide fragment extending between BamHI and HindIII restriction sites of said LCR and an HS4-spanning nucleotide fragment extending between BamHI and BanII restriction sites of said LCR. In yet another embodiment, the HS2 spanning-fragment extending between BstXI and SnaBI restriction sites of said LCR consists of about nucleotides 16241 to 17093 of the sequence of GenBank Accession No. NG_{—}000007.3 (NG7). In still another embodiment, the HS3 spanning-fragment extending between BamHI and HindIII restriction sites of said LCR consists of about nucleotides 12066 to 13360 of the sequence of GenBank Accession No. NG_{—}000007.3 (NG7). In yet another embodiment, the HS4 spanning-fragment extending between BamHI and BanII restriction sites of said LCR consists of about nucleotides 8497 to 9576 of the sequence of GenBank Accession No. NG_{—}000007.3 (NG7).

In one embodiment, the heterologous protein is a globin or Factor IX. In another embodiment, the globin is a β-globin. In a specific embodiment, the β-globin is a human β-globin. In another embodiment, the globin is a γ-globin. In another embodiment, the globin is an α-globin. In yet another embodiment, the globin is a mutant globin. In still another embodiment the globin is a wild-type globin. In another embodiment, the globin is encoded by a nucleotide sequence that has at least 1 intron comprising interfering RNA. In a specific embodiment, the interfering RNA is an antisense RNA, a short hairpin RNA, an siRNA or a microRNA. In another specific embodiment, the interfering RNA selectively degrades a messenger RNA transcript of an undesired host cell gene. In a further embodiment, the undesired host cell gene contains a mutation. In another embodiment, the recombinant vector provides expression of greater than two copies of globin per cell when introduced into a mammal in vivo.

In another embodiment, the heterologous protein is a clotting factor, enzyme, hormone, growth factor, anti-angiogenic factor, antibody or antigen.

In one embodiment, the vector is a lentiviral vector. In another embodiment, the vector is HIV-1-derived. In still another embodiment, the vector is T9, T10, S9, S10, T12, or V9.

In one embodiment, the recombinant vector further comprises a nucleotide sequence encoding a dihydrofolate reductase. In a specific embodiment, the dihydrofolate reductase is a human dihydrofolate reductase.

In another embodiment, the recombinant vector further comprises a central polypurine tract (cPPT) element, a human phosphoglycerate kinase promoter driving a dihydrofolate reductase cDNA (hPGK-DHFR) cassette, a deletion of the 3′ U3 LTR region, or any combination thereof.

In yet another embodiment, the HS-spanning fragments are HS3 and HS4-spanning fragments and 2 GATA-1 binding sites are present at the junction between the HS3 and HS4-spanning fragments.

In one embodiment, the invention provides a method of treating a disorder in a subject comprising administering to a subject in need thereof a therapeutically effective amount of a recombinant vector of the invention, thereby treating the disorder in the subject. In a further embodiment, the recombinant vector of the invention is a lentivector that is used to transduce a hematopoietic progenitor or stem cell. In a specific embodiment, the hematopoietic progenitor or stem cell is transduced in vitro or in vivo. In a particular embodiment, the lentivector has a selectable marker, and a selection step using an anti-folate is additionally performed. In another embodiment, the disorder is a hemoglobinopathy. In a specific embodiment, the hemoglobinopathy is hemoglobin C disease, hemoglobin sickle cell disease (SCD), sickle cell anemia, hereditary anemia, thalassemia, β thalassemia, thalassemia major, thalassemia intermedia, α-thalassemia, or hemoglobin H disease. In a particular embodiment, the disorder is a factor IX deficiency. In a further embodiment, the vector is administered to hematopoietic stem cells of the subject. In various embodiments, the subject is a mammal. In a specific embodiment, the subject is human.

In one embodiment, the invention provides a pharmaceutical composition comprising a recombinant vector of the invention and a pharmaceutically acceptable carrier or excipient. In another embodiment, the invention provides a packaged pharmaceutical comprising a recombinant vector of the invention and associated instructions for using said vector to treat a disorder in a subject.

In one embodiment, the invention provides a method of producing a heterologous protein, the method comprising transfecting a cell with a recombinant vector of the invention, and expressing the heterologous protein in the cell, thereby producing the heterologous protein. In another embodiment, the cell is a hematopoietic progenitor or stem cell. In another embodiment, the method further comprises the step of isolating the protein from the cell.

Other features and advantages of the invention will be apparent from the detailed description, and from the claims.

### DEFINITIONS

As used herein, the term “recombinant lentiviral vector” refers to an artificially created polynucleotide vector assembled from a lentiviral-vector and a plurality of additional segments as a result of human intervention and manipulation.

By “globin nucleic acid molecule” is meant a nucleic acid molecule that encodes a globin polypeptide. Expressly included in this definition are regulatory sequences upstream and/or downstream of the coding sequence. See, for example, GenBank Accession No. NG_{—}000007.3.

By “globin polypeptide” is meant a protein having at least 85% amino acid sequence identity to a human alpha, beta or gamma globin. The sequence of an exemplary beta globin polypeptide is provided at NCBI Accession No. NP_{—}000509.

The term “functional globin gene” refers to a nucleotide sequence the expression of which leads to a globin that does not produce a hemoglobinopathy phenotype, and which is effective to provide therapeutic benefits to an individual with a defective globin gene. The functional globin gene may encode a wild-type globin appropriate for a mammalian individual to be treated, or it may be a mutant form of globin, preferably one which provides for superior properties, for example superior oxygen transport properties. The functional globin gene includes both exons and introns, as well as globin promoters and splice donors/acceptors.

Nucleic acid molecules useful in the methods of the invention include any nucleic acid molecule that encodes a globin polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. ”

By “agent” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “disease” is meant any condition or disorder that damages or interferes with the normal function of a cell, tissue, or organ.

By “an effective amount” is meant the amount of a required agent or composition comprising the agent to ameliorate the symptoms of a disease relative to an untreated patient. The effective amount of composition(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.”

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “detectable label” is meant a composition that when linked to a molecule of interest renders the latter detectable, via spectroscopic, photochemical, biochemical, immunochemical, or chemical means.

By “isolated nucleic acid molecule” is meant a nucleic acid (e.g., a DNA) that is free of the genes which, in the naturally-occurring genome of the organism from which the nucleic acid molecule of the invention is derived, flank the gene.

“Obtaining” as in obtaining a compound refers to purchasing, synthesizing or otherwise acquiring the compound.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. Such siRNAs are used to downregulate mRNA levels or promoter activity.

By “transformed cell” is meant a cell into which (or into an ancestor of which) has been introduced, by means of recombinant DNA techniques, a polynucleotide molecule encoding (as used herein) a polypeptide of the invention.

By “transgene” is meant any piece of DNA that is inserted by artifice into a cell and becomes part of the genome of the organism that develops from that cell

### BRIEF DESCRIPTION OF THE DRAWINGS

FIG. 1. Effect of promoter size of β-globin transgene expression in MEL cells. (A) Top: Schematic representation of lentiviral vectors used in this study. The human β-globin transgene (gene, promoter and 3′ enhancer) is represented in red. The locus control region (LCR) elements HS2 and HS3 are indicated in green and HS4 in blue. The triangle above the 3′LTR indicates deletion in the U3 region making it a self-inactivating (SIN) vector. Middle: Human β-globin promoter elements and major proteins known to interact with them.^{33, 86, 105} Bottom: schematic representation of the different promoters used in this study. R: 130 bp promoter; S: 265 bp promoter; T: 615 bp promoter; V: 1555 bp promoter. (B) Quantification of vector expression in MEL cell pools. Data are shown as expression on mRNA level [Huβ/(Huβ+Muβ)] normalized to vector copy number (VCN). p values were calculated using Student's r-test. n values indicate the number of independent cell pools tested. Abbreviations: LTR: long terminal repeat; SD: splice donor; SA: splice acceptor; Ψ: packaging region; RRE: rev-response element; cPPT: central polypurine tract; hPGK-DHFR: human phosphoglycerate kinase promoter driving the dihydrofolate reductase gene.

FIG. 2. Effect of HS1 on transgene expression in MEL cells. (A) Schematic representation of vector pairs used to evaluate the effect of 5′HS1 element on transgene expression. The orange box indicates the HS1 element. Numbers between vector pairs indicate the size of promoter utilized in each vector. (B) Quantification of vector expression in independent MEL cell pools. Expression at the RNA level (Huβ/(Huβ+Muβ) is normalized to vector copy number (VCN). p values were calculated using Student's r-test. n values indicate the number of independent cell pools.

FIG. 3. Effect of spacing between the transcription start site and LCR2-3-4 on transgene expression. (A, C) Vector constructs used to evaluate the effect of spacing between the transcription start site (TSS) and the LCR(HS2, 3 and 4) on transgene expression. Only globin cassette (Red box) and LCR element (Orange, Green and Blue boxes) are shown (see FIG. 1A for complete vector structure). Spacer DNA (Brown box) was introduced to account for differences in promoter length (A) or to account for the presence of HS1 (C) To avoid introduction of cryptic splice sites or polyA signals to the vector the G6PD cDNA sequence was used in the same orientation relative to transcription as in the genomic locus. The ((S→T))9PD vector contains a spacer sequence of 350 bp, to account for the difference between T (615 bp) and S (265 bp) promoters. The spacers in ((S→V))9PD and (T→V)9PD vectors account for differences between S(265 bp) and V(1555 bp), and T(615 bp) and V(1555 bp) promoters and are 1290 bp and 940 bp, respectively. The T(9→10)PD and S(9->10)PD vectors contain spacer DNA in place of HS1 element (1090 bp). The vectors in the upper panel in (C) contain T (615 bp) promoter while the vectors in lower panel contain the shorter S (265 bp) promoter. (B, D) β-globin expression in MEL pools transduced with the vectors shown in (A) and (C). Results are represented as (Huβ/(Huβ+Muβ) mRNA normalized to vector copy number (VCN). P values were calculated using Student's t-test. n values indicate the number of independent cell pools studied. (E) Effect of the distance between the β-globin transgene TSS and the LCR HS2 on transgene expression. Red boxes represent the human β-globin gene+promoter; orange boxes represent HS1; brown boxes indicate sequence spacer; green boxes represent HS2. The red bars in the graph indicate the mean expression level for a given TSS/HS2 distance.

FIG. 4. Evaluation of the effect of HS4 on transgene expression in MEL cells. (A) Vectors used to address the effect of truncation of HS4 flanking regions on transgene expression. Same symbols as in FIG. 3. The red triangle indicates the truncation site. (B) Quantification of β-globin mRNA expression (Huβ/(Huβ+Muβ) normalized to vector copy number (VCN) in independent MEL cell pools. n values indicate the number of independent cell pools. (C) Vector constructs used to study the 5′flanking region of LCR HS4. The dark blue box indicates the core of 5′HS4. The 5′flanking region was truncated (dotted box in T12PD), replaced with spacer DNA of same size (brown box in T12JseqPD), a fragment of HS3 flanking region of same size (green box in T12HS3PD), or the human IFN-β S/MAR element (light green box) in reverse (T12SAR1PD) or forward (T12SAR2PD) orientation. Throughout the course of the experiment, all vectors were tested for stability by Southern blot. The only vector found to undergo rearrangements was the T12HS3PD vector (data not shown). Only three MEL pools showed no signs of rearrangements and were used in the experiment. (D) Data obtained from comparison of vectors shown in (C) on mRNA level normalized to VCN. p values were calculated using Student's f-test. p*<0.001; p**=0.716.

FIG. 5. Quantitative analysis of therapeutic responses in β-thalassemic mice. (A) Vectors used in in vivo study. The red triangle indicates truncation of 5′flanking region of 5′HS4. For in vivo studies, hPGK-DHFR was either replaced with hPGK-GFP or removed. (B) Long term stability of vector copy number assayed by TaqMan analysis. Three sample mice for each group are shown. (C) Human β-globin transgene mRNA expression in peripheral blood (PB) shown as fraction of total β-globin mRNA and normalized to vector copy [(Huβ/(Huβ+Muβ)/VCN]. For each vector, bars indicate time point during the experiment—in order from left: Week 6, 12, 17, 23, 29, and 37. The number of mice ranged between 8 and 20 per group (total: 99 mice). NT: not tested. p*=0.922; p**=0.012; p***<0.001. (D) Cellulose acetate gel electrophoresis shows Hbhu (mα2:hβ2) levels in vector-transduced bone marrow chimeras 23 weeks after transplantation. Control lanes contain normal C57BL/6 (B6) or HbbTh3/+ (Th) blood samples. The fraction of Hbhu relative to total hemoglobin (Hbhu/Hbhu+Hbmu) and vector copy number are indicated below each sample.

FIG. 6. Quantitative analysis of therapeutic responses in β-thalassemic mice: Relationship between vector copy number and degree of correction of anemia. (A) The transgene expression on mRNA level in PB normalized to vector copy number (Huβ/(Huβ+Muβ)/VCN). The n value indicates the number of mice in each group. Average vector copy number (VCN) for each group is shown in (B). (C) Total Hb level [g/dL] in peripheral blood (PB). (D) Correlation between human β-globin transgene expression on RNA level (Huβ/Huβ+Muβ) and ΔHb. ΔHb level was obtained by subtracting Th3/+Hb ((C) Th3/+=8.9 g/dL)) from total Hb level for each animal and corroborated by acetate gel electrophoresis (data not shown). (E) Correlation between ΔHb (as explained in D), and provector copy number. Each square represents a single animal. The larger dots represent the average for each group, plus or minus s.d. Data collected 23 weeks post bone marrow transplantation. See inset for color-coding of each vector.

FIG. 7 is a schematic diagram showing the structure of the human beta globin gene locus and the TNS9 vector.

, FIG. 8 is a schematic diagram showing the HS1 element of the human β-globin locus control region locus control region (LCR).

FIG. 9 shows the constructs used to evaluate the HS1 element.

FIG. 10 is a schematic diagram showing the strategy used to conduct the experiments described herein.

FIG. 11 is a graph showing the in vitro evaluation of HS1.

FIG. 12 is a construct comparison in MEL clones.

FIG. 13 shows a construct comparison carried out in MEL clones.

FIG. 14 is a schematic diagram showing in vivo experimental design.

FIGS. 15A and 15B are graphs showing an in vivo comparison of various vectors.

FIG. 16 compares in vitro and in vivo data.

FIGS. 17A-17C show Lentiviral vectors, (A) RT9-hFIX-SI and (B) RT10-hFIX-SI, and (C) HMBA-induced human FIX expression in transduced MEL cells.

### DETAILED DESCRIPTION OF THE INVENTION

The invention provides compositions and methods for the treatment or prevention of disorders including, for example, (β-thalassemia, anemias (e.g., sickle cell anemia) and other hemoglobinopathologies. The invention is based, at least in part, on the observation that a β-globin HS1 element significantly increased average β-globin transgene expression in vivo. The expression from a T10 vector, harboring β-globin promoter (615 bp) and LCR containing an HS1 element, was significantly higher than from a T9 vector, lacking the HS1 element. The same results were obtained when S10 vector harboring HS1 and 265 bp promoter was compared to S9 vector with the same promoter but lacking HS1 element. Addition of the locus control region HS1 element had either no or negative effect on transgene expression in vitro, whereas it significantly increased expression in vivo.

Accordingly, the present application provides compositions and methods for erythroid-specific, regulated, high-level and sustained transgene expression. As reported in more detail below, the inventors have demonstrated that a TNS9 lentiviral vector harboring an optimized combination of LCR elements HS 2, 3 and 4 with the β-globin gene promoter from position −615 and the 3′ β-globin enhancer yields tissue specific and therapeutic β-globin expression in thalassemic mice. Four months after transplantation, mice carrying on average 0.5 to 1.0 vector copies in peripheral blood cells showed a Hb levels of 11-13 g/dL, in comparison to 8.0-8.5 g/dL in age-matched controls (May et al 2000). These findings demonstrated that viral-mediated gene transfer could achieve major therapeutic benefit in disorders such as β-thalassemia and paved the way for the use of lentiviral vectors harboring transgenes for treatment in a clinical setting.

### Globin Sequences

Globin genes are well known in the art. Coding and non-coding regions of the gene encoding a hemoglobin subunit beta are described, for example, by Marotta et al., Prog. Nucleic Acid Res. Mol. Biol. 19, 165-175 (1976), Lawn et al., Cell 21 (3), 647-651 (1980), and Sadelain et al., PNAS. 1995; 92:6728-6732, each of which is incorporated herein in it's entirety. An exemplary nucleic acid sequence for the Homo sapiens beta globin region on chromosome 11 is provided at NG_{—}000007. The sequence information and location of the restriction sites that delineate the Dnase I hypersensitive sites HS1, HS2, HS3, and HS4 within the Locus Control Region (e.g., the SnaBI and BstXI restriction sites of HS2, the HindIII and BamHI restriction sites of HS3, and the BamHI and BanII restriction sites of HS4) are also known in the art. See, GenBank Accession Number NG_{—}000007 for exemplary sequence information.

The sequence and position of HS1 is described, for example, by Pasceri et al., “5′HS1 and the Distal {beta}-Globin Promoter Functionally Interact in Single Copy {beta}-Globin Transgenic Mice.” Ann NY Acad. Sci. 1998; 850:377-381; Pasceri et al., “Full Activity From Human beta-Globin Locus Control Region Transgenes Requires 5′HS1, Distal beta-Globin Promoter, and 3′beta-Globin Sequences.” Blood. 1998; 92:653-663; and Milot et al., “Heterochromatin Effects on the Frequency and Duration of LCR-Mediated Gene Transcription,” Cell. 1996; 87:105-114, each of which is incorporated herein by reference in its entirety.

In particular, the HS2 region extends from position 16,671 to 17,058 of the Locus Control Region. The SnaBI and BstXI restriction sites of HS2 are located at positions 17,093 and 16,240, respectively. The HS3 region extends from position 12,459 to 13,097 of the Locus Control Region. The BamHI and HindIII restriction sites of HS3 are located at positions 12,065 and 13,360, respectively. The HS4 region extends from position 9,048 to 9,713 of the Locus Control Region. The BamHI and BanII restriction sites of HS4 are located at positions 8,496 and 9,576 respectively.

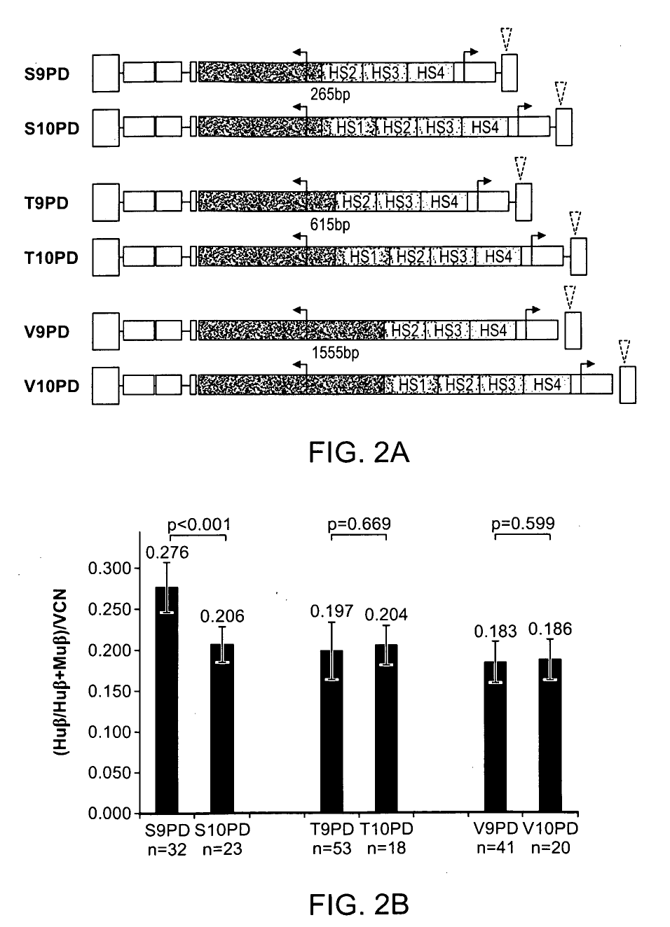

In addition to GenBank Accession Number NG_{—}000007, discussed herein, the following references, which further exemplify the sequences of functional globin genes, are provided below. References 1-4 relate to alpha-type globin sequences and references 4-12 relate to beta-type globin sequences (including beta and gamma globin sequences).
(1) GenBank Accession No. Z84721 (Mar. 19, 1997): This reference describes a 43,058 bp nucleotide sequence encompassing the human genomic sequence of the alpha globin gene cluster, including alpha globin 1 (33234-34311), alpha globin 2 (37038-38118). The sequence presumably contains exons and introns, globin promoters and globin splice donor/acceptor sites as it represents a contiguous genomic DNA sequence.
(2) GenBank Accession No. NM_{—}000517 (Oct. 31, 2000): This reference describes a 575 bp human mRNA alpha globin 2 sequence.
(3) Hardison et al., J. Mol. Biol. (1991) 222(2):233-249. This reference reports a contiguous nucleotide sequence of 10,621 bp (indicated on page 237 as GenBank Accession No. M35026) of the mouse alpha globin gene cluster. This reference also reports an alignment with the corresponding human sequence (page 240). The sequence presumably contains exons and introns, globin promoters and globin splice donor/acceptor sites as it represents a contiguous genomic DNA sequence.
(4) A Syllabus of Human Hemoglobin Variants (1996), by Titus et al., published by The Sickle Cell Anemia Foundation in Augusta, Ga. (available online at http://globin.cse.psu.edu). This reference describes “a comprehensive listing of all known human hemoglobin variants, including variants of the alpha-, beta-, gamma-, and delta-globin chains.” In particular, this reference describes globin mutants having superior oxygen transport properties.
(5) GenBank Accession No. J00179 (Aug. 26, 1993). This reference describes a human 73,326 bp nucleotide sequence encompassing “all of the known beta genes in the cluster on chromosome 11” (see page 8). This genomic DNA region contains the beta and gamma globin genes, and presumably exons, introns, globin promoters and globin splice donor/acceptor sites. The reference specifically indicates that the beta globin promoter is present 5′ upstream of the beta gene (see page 11 of 40, line 14). In addition, the reference specifically indicates the presence of an exon/intron border in the beta gene (see page 11 of 40, line 33).
(6) Tagle et al., Genomics (1992) 13(3):741-760. This reference discloses the 41,101 bp nucleotide sequence of the beta globin gene cluster of a prosimian (see FIG. 2). The reference further discloses results of alignments with corresponding regions from human, rabbit and mouse. The sequence presumably contains exons and introns, globin promoters and globin splice donor/acceptor sites as it represents a contiguous genomic DNA sequence.
(7) Grovsfeld et al., Cell (1987) 51(6):975-985. This reference describes the structure of the human beta globin gene cluster. It further includes a description of the regulatory regions required for appropriate expression of the beta globin gene, including upstream and downstream promoters and enhancers (see page 975, left column, last paragraph through right column, first paragraph).
(8) Li et al., Blood (1999) 93(7):2208-2216. This reference discloses vectors used for testing and optimizing expression cassettes for physiologic human beta globin expression. The report describes the construction of a variety of vectors using different portions or regions of the gamma and beta promoters to drive expression (see Methods and Materials: plasmid constructs). The report also states that “the promoters from the gamma and beta globin genes have been intensively studied for many years” (see page 2214, first column, first paragraph of discussion).
(9) Gorman et al., J. Biol. Chem. (2000) 275(46):35914-35919. This reference reports on the use of ribonucleoprotein particles as carriers for antisense repair of faulty splicing of target genes. In particular, the reference tests the approach using the human IVS2-705 pre-mRNA (a known thalassemic beta-globin pre-mRNA which is defective in splicing). Cell lines are used which stably express the IVS2-705 pre-mRNA (see Methods and Materials).
(10) Slightom et al., Cell (1980) 21(3):627-638. This paper reports the complete nucleotide sequence of human gamma-G and gamma-A beta-type globin genes, including the promoter TATA box region 5′ to the transcription start site (see sequences on page 631).
(11) Fritsch et al., Cell (1980) 19(4): 959-972. This reference reports the isolation of the entire human beta-globin chromosomal region and its fine detail mapping of each of the fetal and adult beta-type genes and their intergenic regions (see page 959, right column, last paragraph of introduction).
(12) Marotta et al., J. Biol. Chem. (1977). 252(14):5040-5053 This paper reports the nucleotide sequence of human mRNA beta-globin on page 5047-5048 and the cloning, mapping and sequencing strategies used to obtain same. Each of these references is incorporated herein by reference in its entirety.

An exemplary amino acid sequence of hemoglobin subunit beta is provided, for example, at NCBI Accession No. P68871. An exemplary amino acid sequence for beta globin is provided, for example, at NCBI Accession No. NP_{—}000509. Sickle cell anemia and beta thalassemia result from mutation and absence, respectively, of beta globin chain.

### Hemoglobinopathies

Hemoglobinopathies include hereditary disorders of hemoglobin. Examples include αthalassemia, β-thalassemia, and sickle cell disorders. As used herein, the term “hemoglobinopathy” or “hemoglobinopathic condition” includes any disorder involving the presence of an abnormal hemoglobin molecule in the blood. Examples of hemoglobinopathies include, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease). The term “sickle cell anemia” or “sickle cell disease” is defined herein to include any symptomatic anemic condition which results from sickling of red blood cells. Manifestations of sickle cell disease include: anemia; pain; and/or organ dysfunction, such as renal failure, retinopathy, acute-chest syndrome, ischemia, priapism and stroke. As used herein the term “sickle cell disease” refers to a variety of clinical problems attendant upon sickle cell anemia, especially in those subjects who are homozygotes for the sickle cell substitution in HbS. Among the constitutional manifestations referred to herein by use of the term of sickle cell disease are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “sickle cell disease” are acute episodes of musculoskeletal pain, which affect primarily the lumbar spine, abdomen, and femoral shaft, and which are similar in mechanism and in severity to the bends. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia and dehydration, all of which potentiate intracellular polymerization of HbS (J. H. Jandl, Blood: Textbook of Hematology, 2nd Ed., Little, Brown and Company, Boston, 1996, pages 544-545). As used herein, the term “thalassemia” encompasses hereditary anemias that occur due to mutations affecting the synthesis of hemoglobin. Thus, the term includes any symptomatic anemia resulting from thalassemic conditions such as severe or β thalassemia, thalassemia major, thalassemia intermedia, and α-thalassemias, such as hemoglobin H disease.

### Non-Globin Transgenes

High-level, erythroid-specific transgene expression is useful for the treatment of a vast number of disorders extending well beyond the hemoglobinopathies. Red blood cell precursors are a useful cell population in which to express transgenes products that can be secreted into the circulation and thus delivered systemically. An example of such in vivo protein delivery is human Factor IX, a clotting factor that is missing in patients with Hemophilia B, see, e.g., A. H. Chang, et al., Molecular Therapy (2008); doi:10.1038/mt.2008.161, which is herein incorporated by reference. The invention further encompasses using these cells as “factories” for protein secretion. Erythroid cell-based expression can be used for large-scale in vitro production of proteins from erythroid cells derived from HSCs or embryonic stem cell-derived hematopoeitic cells. Unlike liver or muscle cells, one can introduce the vectors of the invention into long-lived hematopoietic stem cells (or their precursors such as embryonic stem cells and induced Pluripotent Stem [iPS] cells). The genetic engineering of a stem cell is well suited to achieve long-term therapeutic benefits. The use of an erythroid-specific expression system offers safety advantages in restricting transgene expression to this late stage of cell differentiation in a single lineage. The erythroid lineage is especially attractive in that the nucleus is extruded soon after the endogenous globin genes or the erythroid-specific vector-encoded transgene is transcriptionally activated.

Proteins that could be expressed in this way include, but are not limited to, the proteins disclosed on pages 18-20 and 23-24 of international patent application publication WO 2007/044627, published Apr. 19, 2007, the entire disclosure of which is incorporated herein by this reference. Exemplary proteins include clotting factors (e.g., Factor IX and Factor VIII); enzymes (e.g., adenosine deaminase, the enzymes affected in lysosomal storage diseases, and apolipoprotein E); hormones and growth factors (e.g., parathyroid hormone and erythropoietin), anti-angiogenic factors to curb tumor progression, humanized or non-humanized antibodies (e.g., with anti-tumor or anti-infectious properties) and antigens, for instance, to induce immunity or immune tolerance, for example antigens involved in diabetes, multiple sclerosis, gastritis or arthritis. Furthermore, the prospect of inducing immune tolerance can be useful to prevent or treat inhibitors generated during clotting factor infusion for hemophilia, organ rejection and autoimmune diseases such as diabetes or rheumatoid arthritis.

### Diagnostic Methods

Common hemoglobin variants (e.g., HbS, HbC, Hb D Punjab, Hb E) represent more than 90% of the abnormal Hbs observed and they may be identified by a simple approach, such as agarose gel isoelectricfocusing (IEF) or cation-exchange HPLC. Rare variants may be identified based on the comparison of the electrophoretic mobility of the mutated hemoglobin under various experimental conditions. When the variant displays a typical matrix of electrophoretic mobility and is observed under the same hematological and clinical context in a population or in a region where it has already been described, the chance for an exact identification is high. In other cases, the measurement of the mass of the globin chains by electrospray analysis may be used, hemoglobin disorders are identified using any method known in the art. For example, the molecular diagnosis of Thalassemia may be accomplished using a PCR-based approach, such as allele-specific oligonucleotide hybridisation (ASO) or allele-specific priming, large deletion mutations may be diagnosed by gap-PCR. If the mutation remains unidentified, a direct DNA sequence analysis may be performed. A conformational probe, denaturing gradient gel electrophoresis (DGGE) or denaturing HPLC (DHPLC) may be used to specify the region of a gene carrying an abnormality.

Exemplary diagnostic techniques are listed in Table 1.

| | | | | |
|---|---|---|---|---|
| ELECTROPHORETIC | Isoelectric focusing | Cellulose acetate | Agar gel | Globin chain |
| STUDIES | | electrophoresis | electrophoresis at pH6 | electrophoresis |
| CHROMATO | Cation-exchange | Reversed-phase | Microcolumn | |
| GRAPHIC | HPLC | HPLC | chromatography | |
| STUDIES | | | | |
| Functional | Stability tests | Oxygen binding | | |
| Studies | | studies | | |
| Molecular | Polymerase chain | ASO | Allele-specific priming | Gap-PCR |
| Biology | reaction (PCR) | hybridization | methods | |
| Studies | | methods | | |
| | Restriction fragment | Restriction | Conformation analysis | |
| | length | enzymes | | |
| | polymorphisms | | | |
| | (RFLP) | | | |
| Characterization | General | Mass | Structural | Characterization |
| of the | considerations | spectrometry | characterization of a | by molecular |
| Molecular | | studies | variant by protein | biology studies |
| Defect | | | chemistry | |

### Polynucleotide Therapy

Patients diagnosed as having thalassemia, anemia (e.g., sickle cell anemia) or another hemoglobinopathology are amenable to treatment with a vector encoding a beta globin protein. Polynucleotide therapy featuring a polynucleotide encoding a β globin protein, variant, or fragment thereof is another therapeutic approach for treating thalassemia, anemias (e.g., sickle cell anemia) and other hemoglobinopathologies or diseases. Such nucleic acid molecules can be delivered to cells of a subject having thalassemia, anemias (e.g., sickle cell anemia) and other hemoglobinopathologies. The nucleic acid molecules must be delivered to the cells of a subject in a form in which they can be taken up so that therapeutically effective levels of beta globin protein or fragments thereof can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an beta globin protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). In one preferred embodiment, a viral vector is used to administer a beta globin polynucleotide to a bone marrow cell, a hematopoietic stem cell, an erythroid cell or an erythroid precursor. Preferably, this administration enhances globin expression in primary erythroid cells.

Non-viral approaches can also be employed for the introduction of therapeutic to a cell of a patient requiring beta globin. For example, a nucleic acid molecule can be introduced into a cell by administering the nucleic acid in the presence of lipofectin (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant beta globin protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

If desired, the recombinant vectors of the invention include large portions of the locus control region (LCR), which include any one or more DNase I hypersensitive sites (e.g., HS1, HS2, HS3 and HS4). In prior studies, smaller nucleotide fragments spanning the core portions of HS2, HS3 and HS4 have been utilized. See, for example, Sadelain et al. Proc. Nat. 7 Acad. Sci. (USA) 92: 6728-6732 (1995); Lebouich et al., EMBO J. 13: 3065-3076 (1994). The term “large portions” refers to portions of the locus control region which encompass larger portions of the hypersensitive sites as opposed to previously tested fragments including only the core elements. The regions may be the complete site or some lesser site which provides the same functionality as the specific sequences set forth below. In preferred embodiments of the invention, the large portions of the locus control regions are assembled from multiple fragments, each spanning one of the DNase I hypersensitive sites. In addition, the locus control region has two introduced GATA-1 binding sites at the junction between HS3 and HS4. While not intending to be bound by any specific mechanism, it is believed that the incorporation of these transcription factor binding sites enhances the effectiveness of the vector.

Additional elements may be included in the vectors of the invention to facilitate utilization of the vector in therapy. For example, the vector may include selectable markers, to confirm the expression of the vector or to provide a basis for selection of transformed cells over untransformed cells, or control markers which allow targeted disruption of transformed cells, and thus the selective removal of such cells should termination of therapy become necessary

The vectors of the invention are used in therapy for treatment of individuals suffering from hemoglobinopathies. In one embodiment of the invention, hematopoietic progenitor or stem cells are transformed ex vivo and then restored to the patient. As used in the specification and claims hereof, the term “hematopoietic progenitor sand stem cells” encompasses hematopoietic cells and non-hematopoietic stem cells, e.g., embryonic stem cells, hematopoietic stem cell precursors, or any of the latter generated by nuclear transfer from a somatic cell. It is know in the art that efficient genes transfer into human embryonic stem cells can be achieved using lentiviral vectors.

Selection processes may be used to increase the percentage of transformed cells in the returned population. For example, a selection marker which makes transformed cells more drug resistant than un-transformed cells allows selection by treatment of the cells with the corresponding drug. When DHFR is used as a selection marker, it can be used for enrichment of transduced cells in vitro, or for in vivo selection to maintain the effectiveness of the vector.

### Therapeutic Methods

The present invention provides methods of treating thalassemia, anemias (e.g., sickle cell anemia) and other hemoglobinopathologies or diseases and/or disorders or symptoms thereof which comprise administering a therapeutically effective amount of a pharmaceutical composition comprising a vector as described herein to a subject (e.g., a mammal such as a human). Thus, one embodiment is a method of treating a subject suffering from or susceptible to a disease (e.g., globin related disease) or disorder or symptom thereof. The method includes the step of administering to the mammal a therapeutic amount of an amount of a composition herein sufficient to treat the disease or disorder or symptom thereof, under conditions such that the disease or disorder is treated.

The methods herein include administering to the subject (including a subject identified as in need of such treatment) an effective amount of a composition described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compositions herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compositions herein may be also used in the treatment of any other disorders in which an insufficiency in hemoglobin or beta globin may be implicated.

In one therapeutic approach, an agent identified as described herein is administered to the site of a potential or actual disease-affected tissue or is administered systemically. The dosage of the administered agent depends on a number of factors, including the size and health of the individual patient. For any particular subject, the specific dosage regimes should be adjusted over time according to the individual need and the professional judgment of the person administering or supervising the administration of the compositions.

In one embodiment, the invention provides a method of monitoring treatment progress. The method includes the step of determining a level of diagnostic marker (Marker) (e.g., any target delineated herein modulated by a composition herein, a protein or indicator thereof, etc.) or diagnostic measurement (e.g., screen, assay) in a subject suffering from or susceptible to a disorder or symptoms thereof associated with thalassemia, anemias (e.g., sickle cell anemia) and other hemoglobinopathologies or diseases, in which the subject has been administered a therapeutic amount of a composition herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In one therapeutic approach, an agent identified as described herein is administered to the site of a potential or actual disease-affected tissue or is administered systemically. The dosage of the administered agent depends on a number of factors, including the size and health of the individual patient. For any particular subject, the specific dosage regimes should be adjusted over time according to the individual need and the professional judgment of the person administering or supervising the administration of the compositions.

### Pharmaceutical Therapeutics

The invention provides compositions useful as therapeutics for the treatment or prevention of thalassemia, anemias (e.g., sickle cell anemia) and other hemoglobinopathologies or diseases. Accordingly, an agent discovered to have medicinal value using the methods described herein is useful as a drug or as information for structural modification of existing compositions, e.g., by rational drug design. Such methods are useful for screening agents having an effect on a variety of conditions characterized by a reduction in beta globin function or expression.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the thalassemia, anemias (e.g., sickle cell anemia) and other hemoglobinopathologies or diseases. Generally, amounts will be in the range of those used for other agents used in the treatment of other diseases associated with a deficiency in hemoglobin expression or activity, although in certain instances lower amounts will be needed because of the increased specificity of the composition. A composition is administered at a dosage that increases beta globin expression as determined by a method known to one skilled in the art, or using any assay that measures the expression or the biological activity of a beta globin polypeptide.

### Formulation of Pharmaceutical Compositions

The administration of a composition for the treatment of thalassemia, anemias (e.g., sickle cell anemia) or other hemoglobinopathologies may be by any suitable means that results in a concentration of the therapeutic that, combined with other components, is effective in ameliorating, reducing, or stabilizing a thalassemia, anemia (e.g., sickle cell anemia) or other hemoglobinopathology. In one embodiment, a vector of the invention or a cell comprising such a vector is contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

In one embodiment, a pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a globin disorder, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compositions is only sparingly or slightly soluble in water, a dissolution enhancing or solubilizing agent can be added, or the solvent may include 10-60% w/w of propylene glycol or the like.

### Kits or Pharmaceutical Systems

The present compositions may be assembled into kits or pharmaceutical systems for use in ameliorating a thalassemia, anemia (e.g., sickle cell anemia) or other hemoglobinopathology. Kits or pharmaceutical systems according to this aspect of the invention comprise a carrier means, such as a box, carton, tube or the like, having in close confinement therein one or more container means, such as vi microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Promoter Length Controls LCR-Regulated β-Globin Expression in Lentiviral Vectors

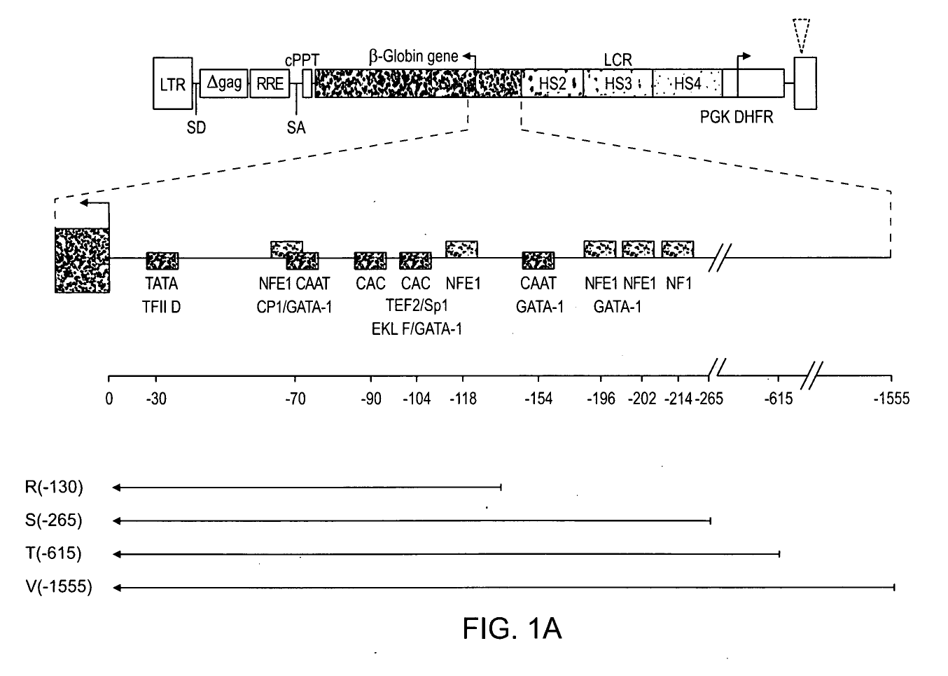

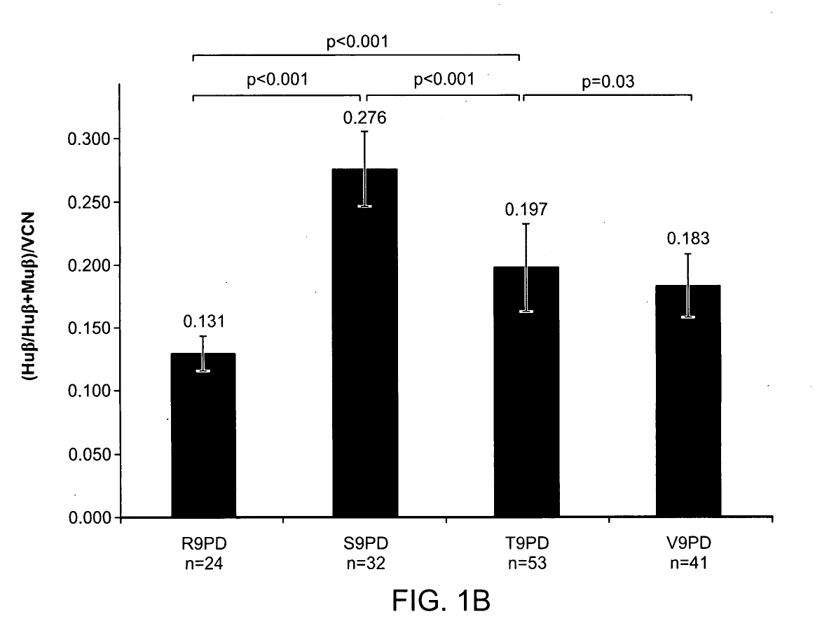

To investigate the effect of β-globin promoter length on lentivirus-encoded β-globin expression, we created a panel of self-inactivating (SIN) lentiviral vectors harboring four promoters of either 130, 265, 615 or 1555 bp (FIG. 1A). All vectors encode the same β-globin transgene, β-globin LCR (spanning DNaseI hypersensitive sites HS2-3-4) and 3′ β-globin enhancer. The 615 bp promoter (termed “T”) has been extensively characterized previously.^{22, 39, 50-53,85} The promoter termed “V” extends to position−1555 and is the same as published by Ellis and colleagues.⁶⁹ The two shorter promoters termed “R” and “S”, start at positions −130 and −265, respectively, and have been utilized by others.^{20,25-30,77} As shown in FIG. 1A, the R promoter extends to, but does not include, the CAAT box present in the −150 region of the human β-globin promoter.⁸⁶ The S (265 bp) promoter includes the CAAT box as well as NF-E1 and NF1 binding sites. Promoters T (615 bp) and V (1555 bp) include less well characterized upstream sequences. These vectors were characterized in murine erythroleukemia (MEL) cells, which do not constitutively express globin genes but do so after induction with hexamethylene bisacetamide (HMBA). We found that average expression level normalized to vector copy number (VCN) were the same for selected and unselected MEL cell pools, as long as the VCN was within detection range. In all studies, vectors were therefore transduced at low multiplicity of infection (moi) and then selected with methotrexate. The VCN in 150 independent pools was between 1.0 and 2.1. Transgene expression was assessed before and after HMBA-induction using primer extension analysis, and then normalized to VCN (FIG. 1B). The R9PD (n=24 pools) vector was the poorest expressing vector in this panel, averaging 15±1.4% of the endogenous murine β-globin. The T9PD and V9PD vectors performed slightly better, averaging 24.5±3.7% (n=53) and 22±2.6% (n=41), respectively, and were significantly weaker than the S9PD vector (n=32 pools), which expressed at 38±3% of endogenous β-globin (p<0.001 relative to T9PD or V9PD). Transgene expression was undetectable for S9PD, T9PD and V9PD vectors in undifferentiated MEL cells, consistent with the expected late stage erythroid specificity imparted by the β-globin promoter. The R9PD vector was not fully differentiation stage-specific, since pre-induction transgene expression was about 10% of its maximum levels. Thus, promoter length dramatically influences LCR function within the confines of a lentiviral vector, spanning a broad 2.5-fold range. All promoters ≧265 bp in length retained differentiation stage-dependent inducibility.

### Example 2

### HS1 does not Increase Average Transgene Expression in MEL Cells

In order to evaluate the possible contribution of the 5′HS1 element to lentivirus-encoded human β-globin expression, we added 5′HS1 to the LCR of S9PD, T9PD and V9PD vectors (FIG. 2A). Addition of 5′HS1 to vectors harboring the T (615 bp) or V (1555 bp) promoters had no effect on average transgene expression per vector copy (p=0.669 and 0.599, respectively, FIG. 2B). In the context of the 265 bp promoter, however, addition of 5′HS1 significantly decreased the average transgene expression per vector copy, from 38±3% (S9, n=32 pools) to 26±2% (S10, n=23) of the endogenous β-globin (p<0.001). None of the vectors expressed transgene in undifferentiated MEL cells (data not shown). Thus, in MEL cells, addition of HS1 has no effect on long promoter constructs and diminishes the performance of the S9PD vector.

### Example 3

### Spacing Between the Proximal Promoter and the HS2-3-4 LCR Determines Globin Transgene Expression Levels

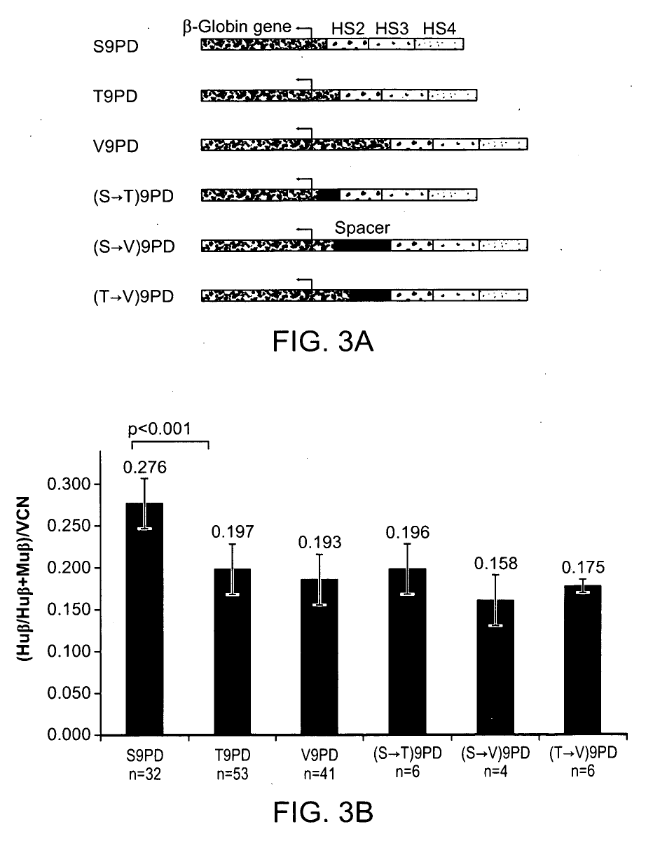

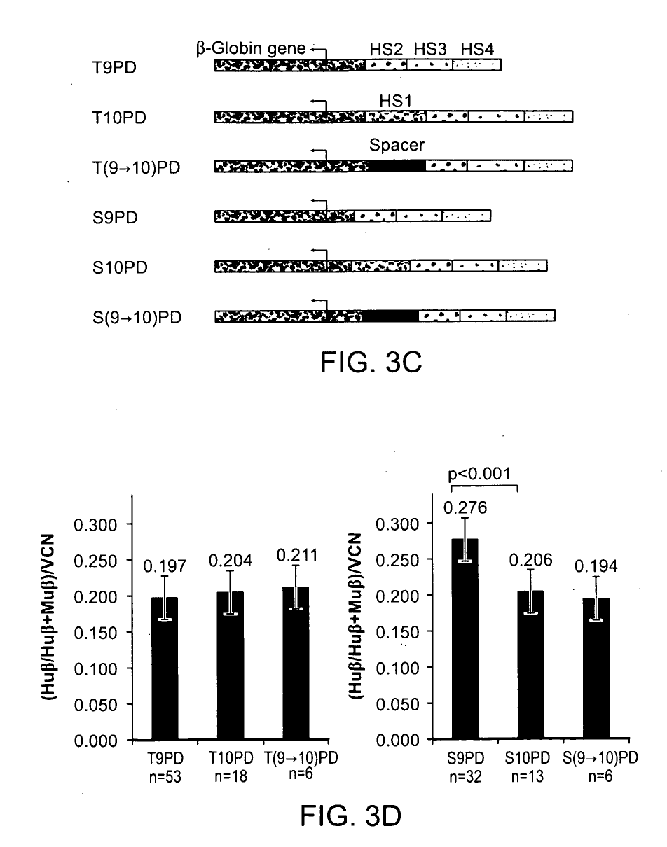

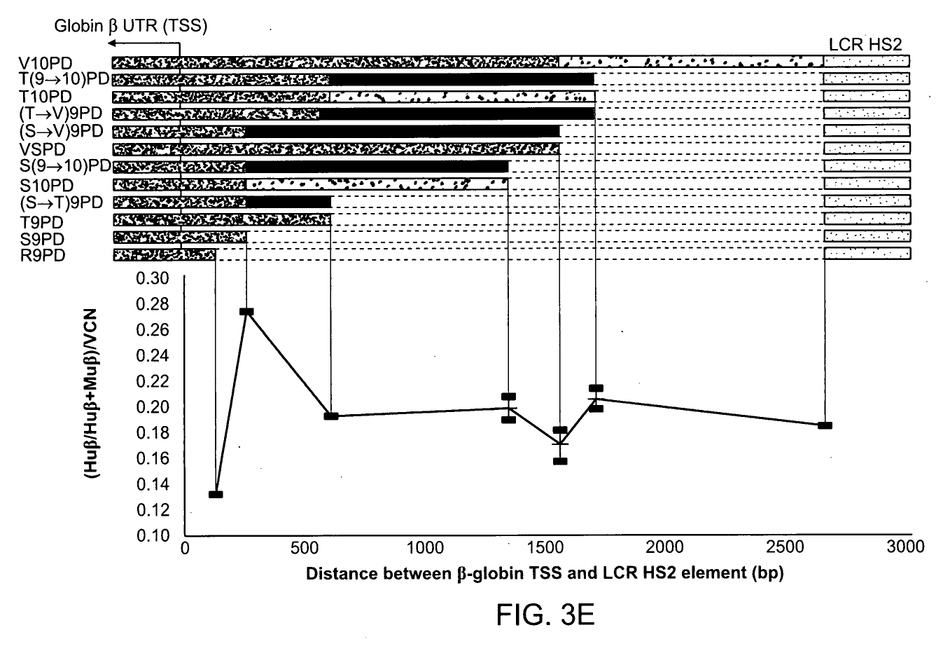

Having observed the higher expression of S9PD relative to T9PD and V9PD (FIG. 1), the decreased expression of S10PD relative to S9PD (FIG. 2), and noting the comparable lengths of the V promoter and 5′HS1 element (1290 bp and 1090 bp additional sequence interposed between the S promoter and the beginning of HS2, FIGS. 3A and C), we hypothesized that promoter/LCR distance may be directly affecting average expression levels in this subset of vectors. To address this possibility, we designed a series of vectors carrying a spacer sequence derived from human G6PD cDNA (FIGS. 3A and C). The addition of a spacer sequence to the S promoter, to extend it to the size of T (S→T) or V (S→V), lowered average expression to levels equal to the corresponding promoters, T and V (FIG. 3B). Addition of a spacer sequence to extend T to the size of V had the same effect, lowering expression of the T vector to that of the V9PD vector (FIG. 3B). These data show that the decrease in the average expression level observed with T (615 bp) or V (1555 bp) promoters, relative to S, is due to the spacing of the β-globin transcription start site (TSS) and LCR(HS2-3-4), rather than specific promoter sequences between position −265 and −1555. Addition of the HS1-spacer to the T9PD vector [T(9-> 10)PD, FIGS. 3C and D], had no effect on average globin transgene expression, which remained comparable to the parental T9PD and T10PD vectors. In the context of the shorter S promoter, however, addition of the spacer [S(9→10)PD] significantly reduced transgene expression to the same level as S10PD (p<0.001). These data are consistent with a lack of function of HS1 in MEL cells and further support that the negative effect of HS1 on S vectors is due to a spacing effect. These findings are summarized in FIG. 3E, in which the exact position and length of all vectors shown in FIGS. 1-3 are annotated. S9PD, which encodes the 265 bp promoter and lacks HS1, provides the most optimal spacing between the globin proximal promoter and LCR elements, resulting in the highest transgene expression level in MEL cells. Shortening the promoter to −130 significantly lowers the expression level from 38±3% (S9PD) to 15±1.4% (R9PD) of the endogenous β-globin, highlighting the importance of elements present between −130 and −265 (FIG. 1A). Increasing the distance between the 265 bp promoter and the HS2-3-4 LCR by either increasing promoter length or interposing HS1 or control sequence uniformly resulted in the same decrease of mean globin expression. These results underscore the importance of regulatory elements placement at a very specific distance from each other in order to achieve optimal functional interactions within therapeutic vectors, and further support the conclusion that HS1 has no measurable activity as part of the LCR holocomplex in MEL cells.

### Example 4

### The Flanking Regions of HS4 Increase Vector-Encoded Globin Expression

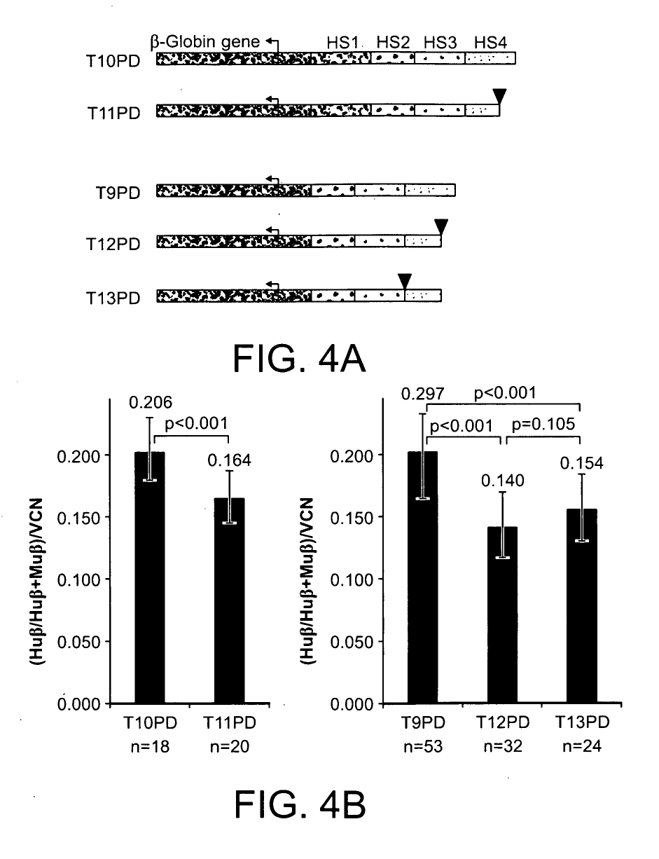

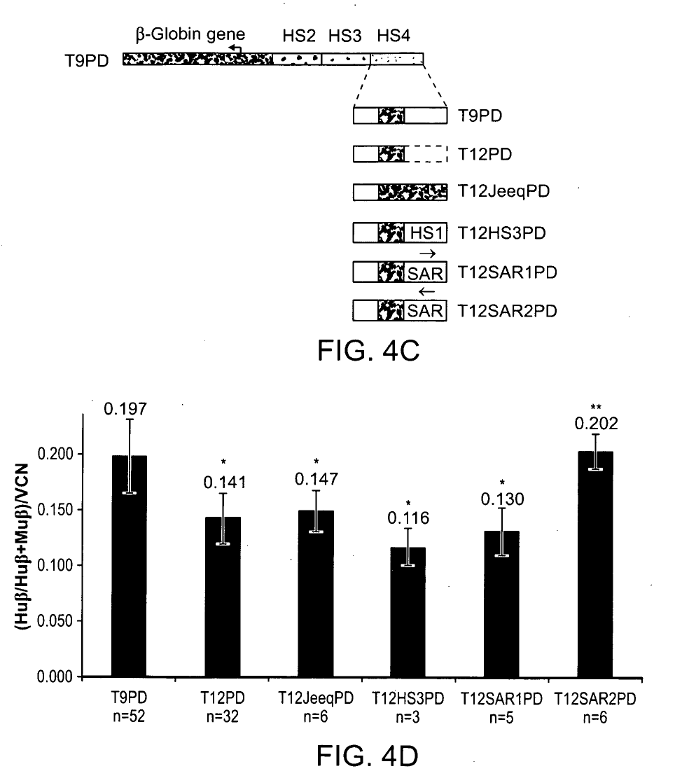

We next examined the contribution of the flanking regions to an HS element, focusing on HS4. To this end, we created the two panels of vectors shown in FIG. 4A. In the first set, we truncated the 5′flanking region of 5′HS4 in the context of the T promoter (615 bp) and LCR1-4 (vector T11PD, n=20 pools). In the second set, we constructed vectors encoding the T promoter and HS2-4, with truncations of the 5′ (vector T12PD, n=32) or 3′ (vector T13PD, n=24) flanking regions of 5′HS4. Truncation of the 5′ flank of 5′HS4 significantly decreased mean globin expression in T11PD, from 26±2.5% to 20±2% of the endogenous β-globin (p<0.001). Similarly, when 5′ or 3′ flanking regions of 5′HS4 were truncated in the vectors lacking HS1, the average expression levels decreased from 24.5±3.7% to 16±3% and 18±3%, respectively (p<0.001). These data suggest that the AT-rich flanking regions of 5′HS4 may play an important role in the overall function of the LCR. To further characterize the role of 5′HS4 we engineered additional vectors (FIG. 4C), in which the 5′ flanking region of HS4 was replaced with an unrelated DNA spacer of the same size (T12JseqPD, n=6), the same size fragment of 5′ flanking region of HS3 (T12HS3PD, n=3), or another A-T rich element, the human IFN-□S/MAR78, 87, cloned in forward (T12SAR2PD, n=6) or reverse (T12SAR1PD, n=6) orientation.

Addition of spacer DNA to the T12PD vector, substituting for the HS4 truncation, did not alter average β-globin expression in MEL cells. No significant change in transgene expression was observed when the truncated fragment of 5′HS4 was replaced with the flanking region of HS3 or the S/MAR sequence in reverse orientation (T12HS3PD and T12SAR1PD vectors) either. In contrast, however, replacement with the human IFN-β S/MAR sequence in forward orientation restored average expression to the level detected for the T9PD vector (FIG. 4D), suggesting that flanking regions play important role in LCR function and do not function solely as spacers between HS cores.

### Example 5

### Both HS1 and HS4 Contribute to Therapeutic Globin Expression In Vivo

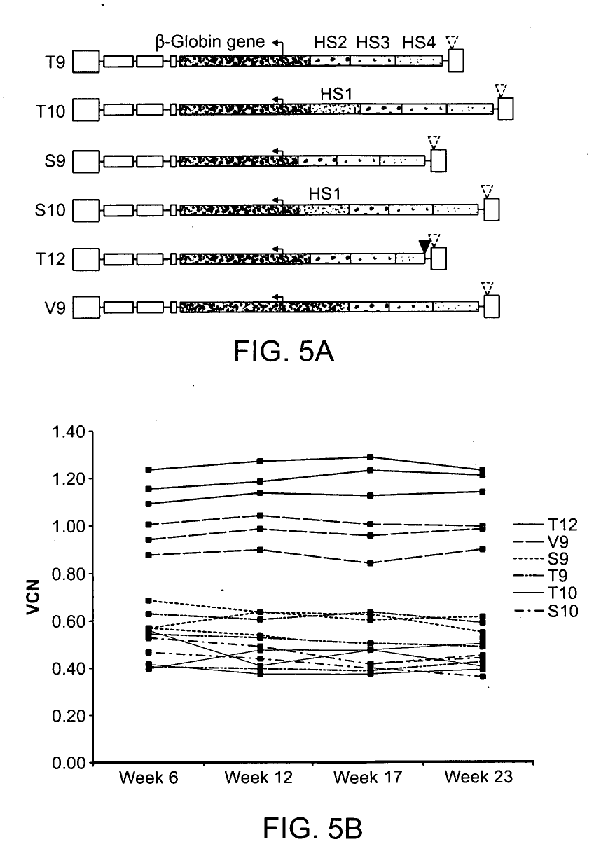

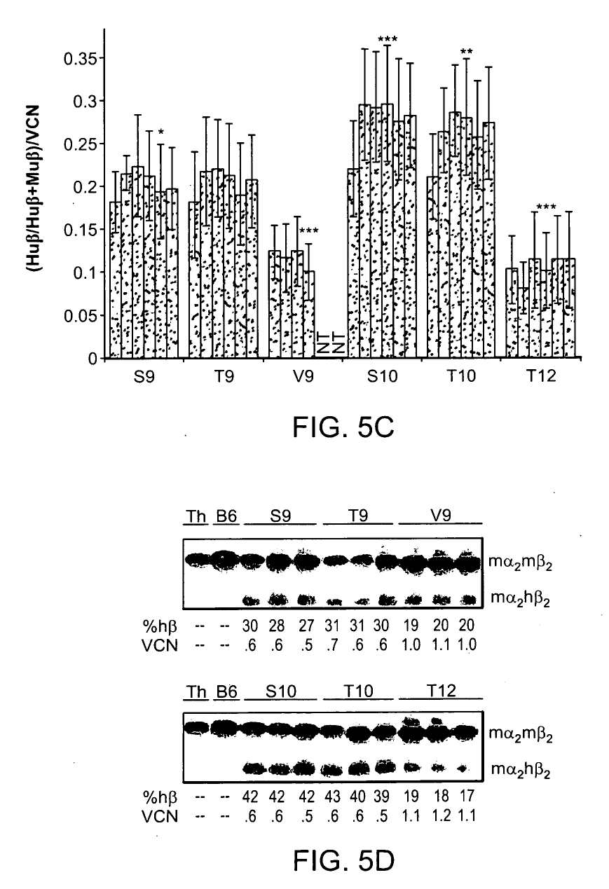

Based on the results from MEL cell studies, we selected the T9, T10, S9, S10, T12 and V9 vectors for in vivo comparison in the murine HbbTh3/+ model of β-thalassemia^{84,88} (FIG. 5A). Bone marrow chimeras were followed for 8 months, including periodic measurements of blood cell counts, VCN, human β-globin transcription, Hb levels and Hb composition. The best vector in MEL cell studies, the S9 vector, did not outperform the reference T9 vector in vivo: the S9 and T9 vectors expressed human β-globin at the same level (FIG. 5C). The V9 vector, which harbors the longer 1555 bp β-globin promoter⁸⁹, performed less well than expected from the MEL cell studies (FIGS. 1B and 2B), as mean expression levels were lower than for T9 (FIG. 5C). Thus, in the context of HS2-3-4, the S and T promoters proved to be equivalent in vivo. The same conclusion applies in the context of HS1-4, as S10 and T10 performed similarly (FIG. 5C).

In stark contrast to data from MEL cell experiments (FIG. 2B), the addition of the 5′HS1 element significantly improved the vectors' performance in vivo (S9 vs. S10 and T9 vs. T10, FIG. 5C). The transgene expression, on mRNA level per vector copy, increased from 27±6% (S9 and T9), of the endogenous β-globin mRNA, to 41±9% (S10 and T10) (p<0.001). These data show that HS1 plays an important role in LCR function within therapeutic vectors, consistent with in vivo deletion studies.^{70,71} Consistent with the MEL findings (FIG. 4) and other in vitro studies 59,62,71, the T12 vector, which encodes a reduced HS4 element, expressed at significantly lower level than T9 (FIG. 5C).

The mRNA data were confirmed on the protein level (FIG. 5D). All animals (n=99) showed sustained amelioration of their anemia, without decrease over the period of observation. The T9 and S9 vectors resulted in similar levels of chimeric hemoglobin (mα2hβ2), on the order of 44-54% per vector copy. Vectors containing 5′HS1 element (S10 and T10) generated higher levels of chimeric Hb, while V9 and T12 expressed much less. These analyses are further detailed below in relation to vector copy number in long-term bone marrow chimeras.

### Example 6

### Impact of Vector Design on the Vector Copy Number Required to Durably Treat Anemia

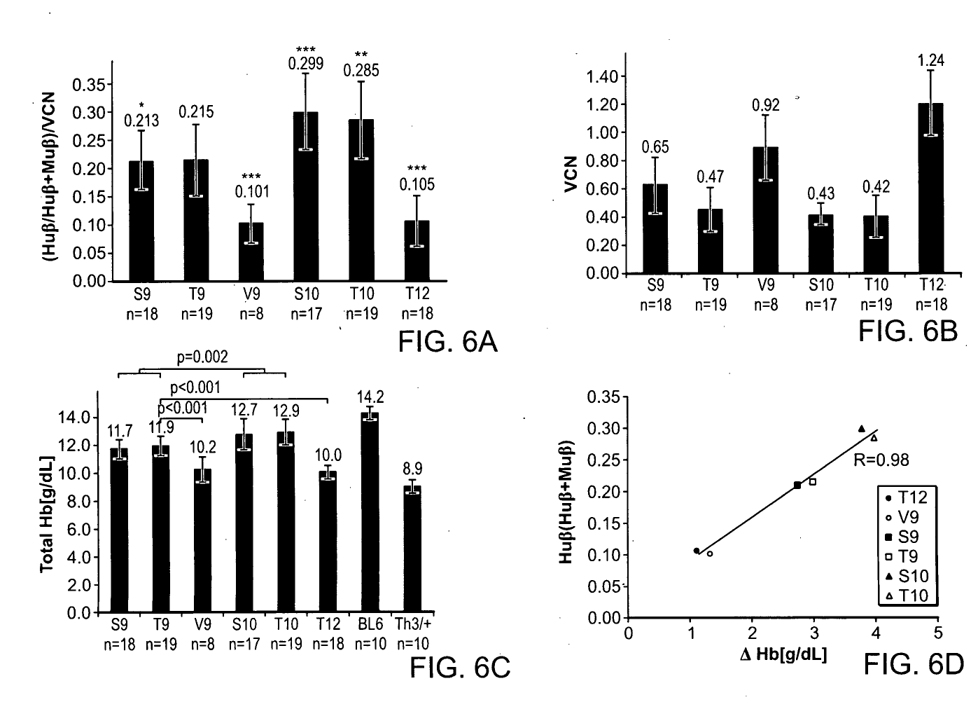

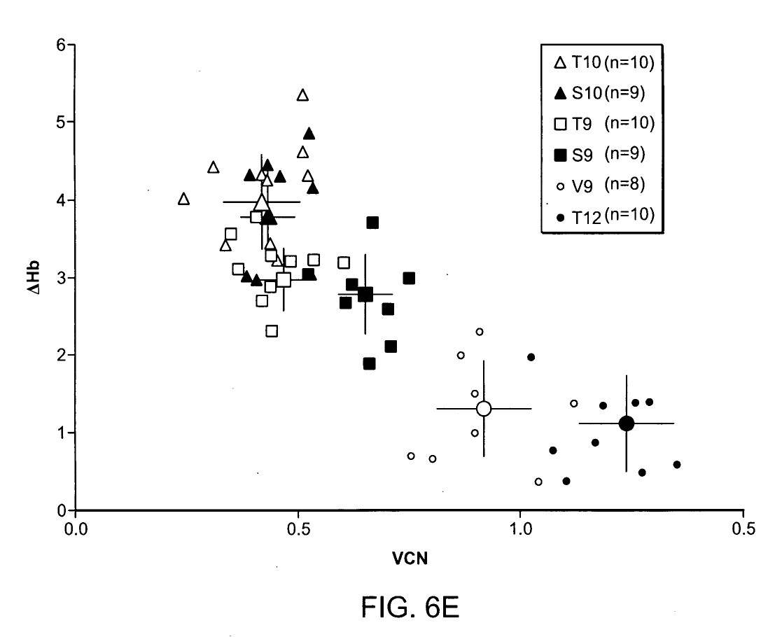

We closely compared the therapeutic efficacy of each vector in 99 long-term bone marrow chimeras, focusing on normalized mRNA and protein levels, and the VCN required to attain durable therapeutic responses in β-thalassemic mice. Normalized expression, 6 months after transplantation, for each vector is shown in FIG. 6A. The vectors containing HS1, S10 and T10, expressed the most human β-globin transcript, followed by S9 and T9 (p<0.001, S10 and T10 vs. S9 and T9). The V9 and T12 vectors performed more poorly (p<0.001, calculated in respect to T9). The vector copy number at the same time point (week 23) is shown in FIG. 6B. The average copy number ranged from 0.42 for T10 and 1.24 for T12. Vector copy was stable throughout the experiment, as illustrated from 3 representative mice per vector in FIG. 5B. We quantified and compared the in vivo function of each vector on the protein level by measuring the total and chimeric Hb level in all treated mice (FIG. 6C). Mice treated with vectors containing the 5′HS1 (T10, n=19 and S10, n=17) exhibited the highest Hb levels: 12.9±0.9 g/dL and 12.7±1.1 g/dL, respectively. When normalized to vector copy, those vectors provide 9.5 g/dL and 8.8 g/dL of Hb per vector copy. The total hemoglobin levels in mice treated with T9 (n=19) and S9 (n=18) were very similar (11.9±0.8 and 11.7±0.6 g/dL, respectively), and significantly lower than for T10 (n=19) and S10 (n=17) (p=0.002). When normalized to vector copy, the T9 and S9 vectors provided about 6.4 g/dL and 4.2 g/dL of human Hb per vector copy, respectively. Addition of HS1 thus nearly doubled the hemoglobin output per vector copy. Again, T12 and V9 vectors performed the least well and mice treated with those vectors exhibited significantly lower Hb levels (10±0.5 and 10.2±0.9 g/dL, respectively; p<0.001, calculated in respect to T9), which translated into 0.9 g/dL and 1.4 g/dL per vector from T12 and V9, respectively (FIG. 6C). Protein expression was directly proportional to β-globin mRNA transcript levels, as shown in FIG. 6D (R2= 0.98). FIG. 6E represents the gain in hemoglobin in relation to vector copy number analyzed in 99 mice. The results indicate that 1-2 vector copies per cell achieve major therapeutic responses with vectors T9, S9, S10 and T10. In contrast, 3-5 copies per cell would be required to treat anemia to the same extent using the V9 and T12 vectors.

### Example 7

### HS1 Element Enhances Globin Expression in Primary Erythroid Cells

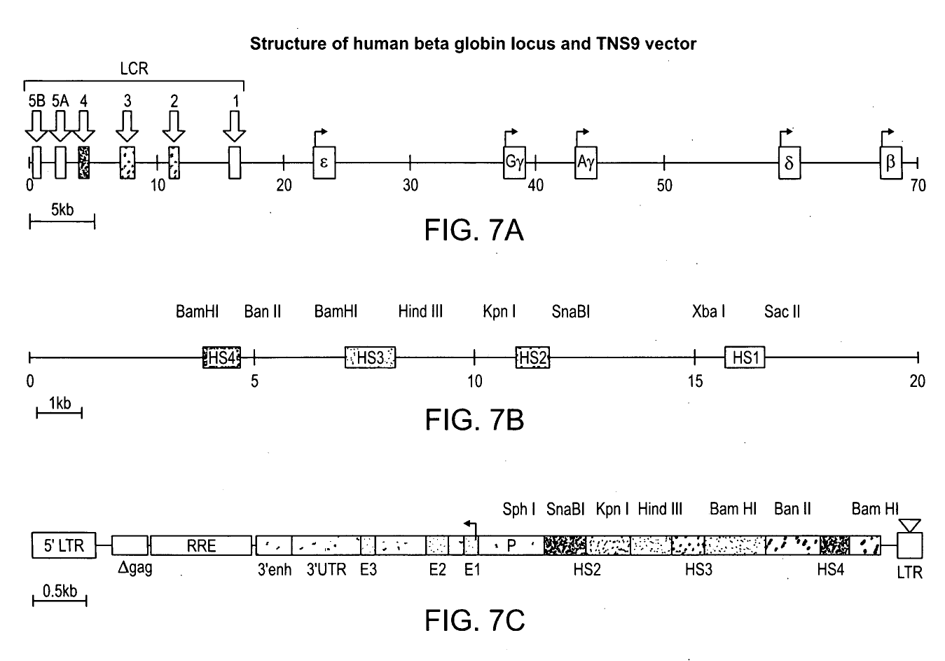

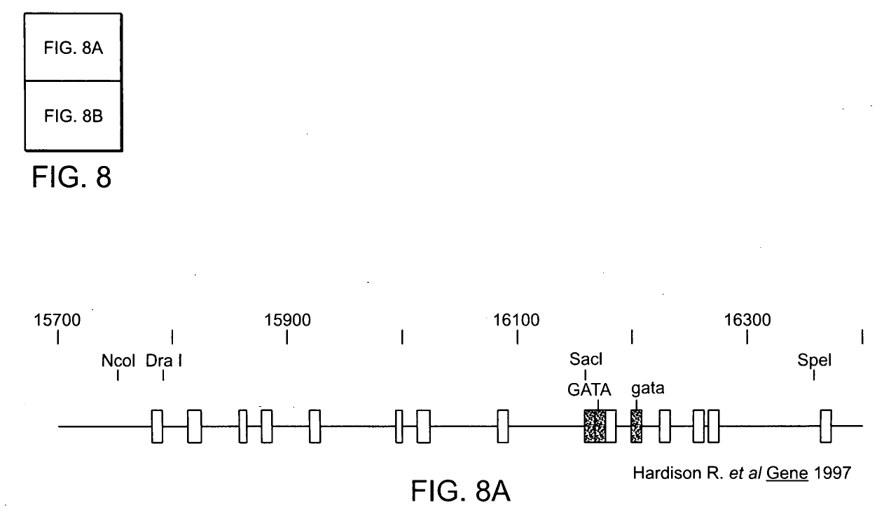

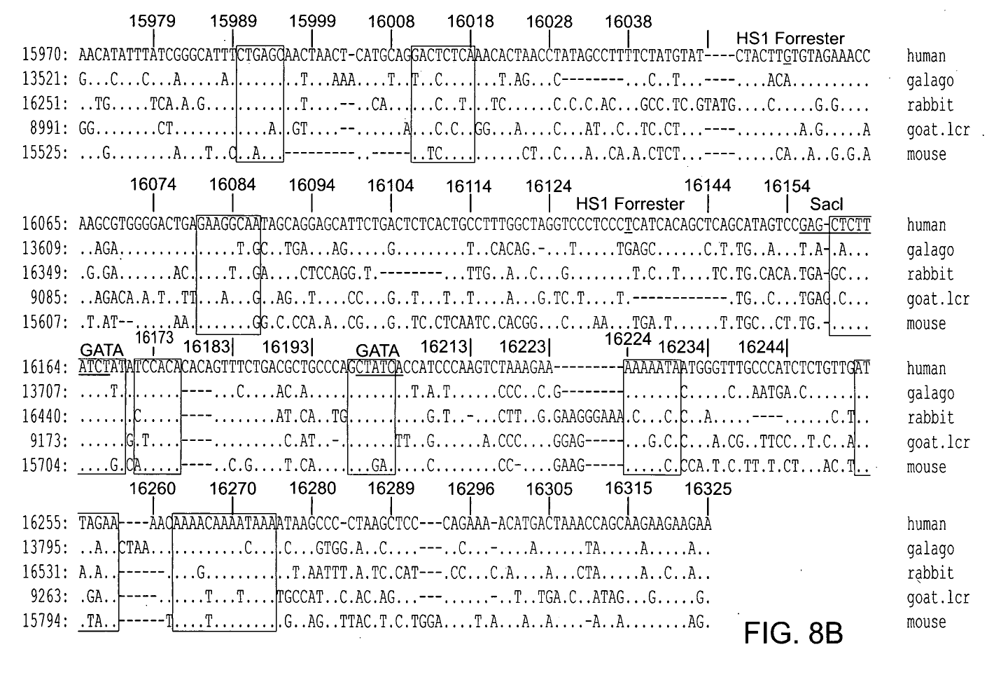

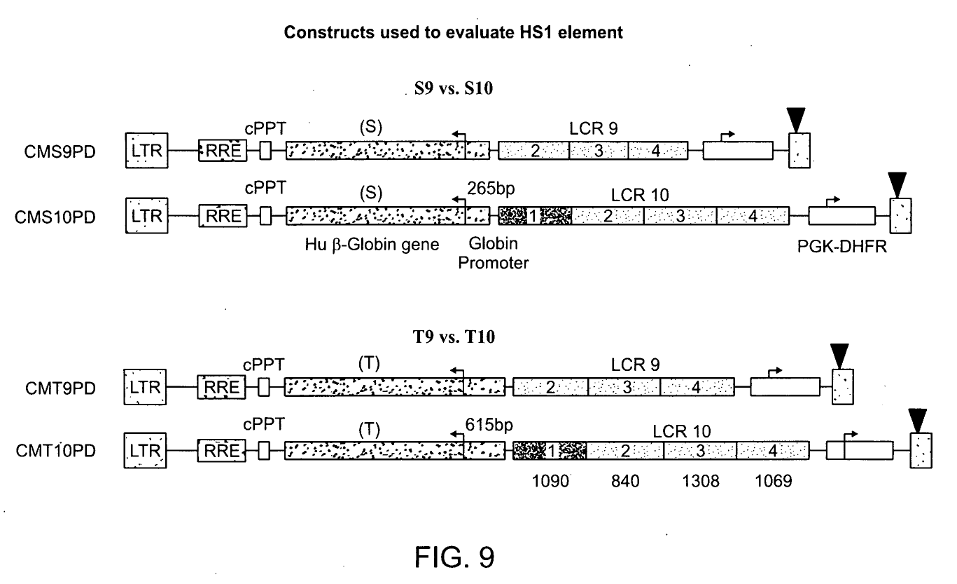

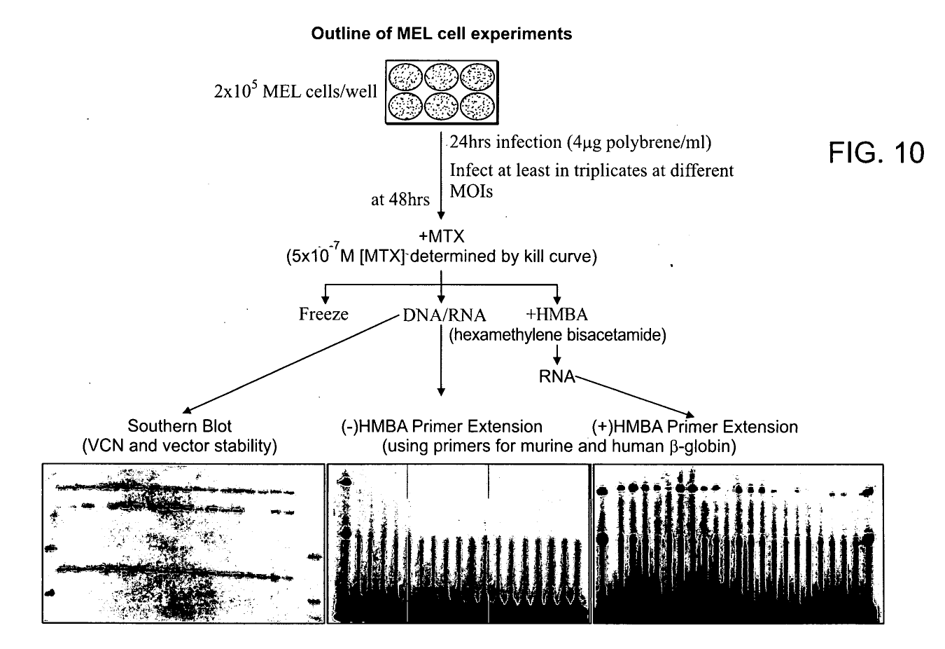

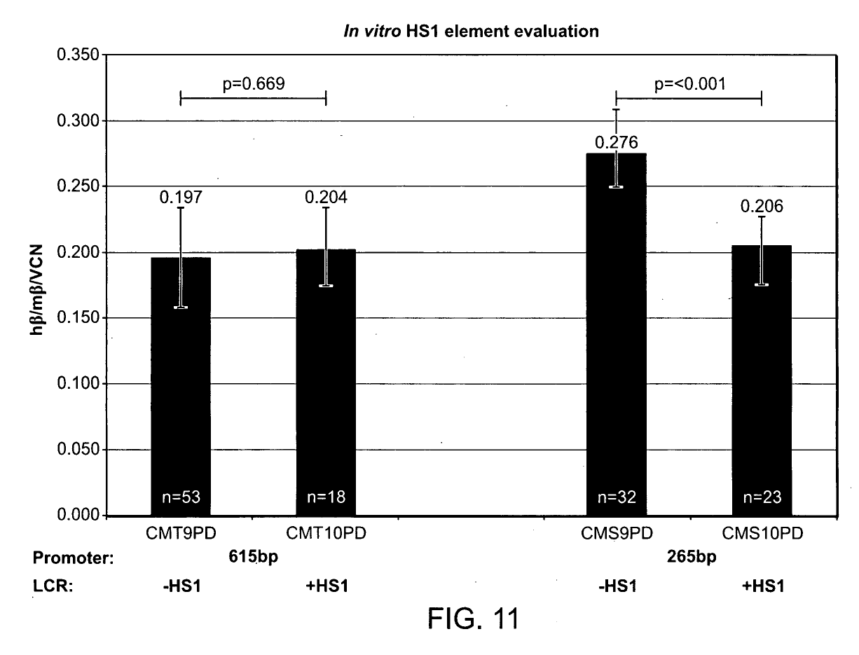

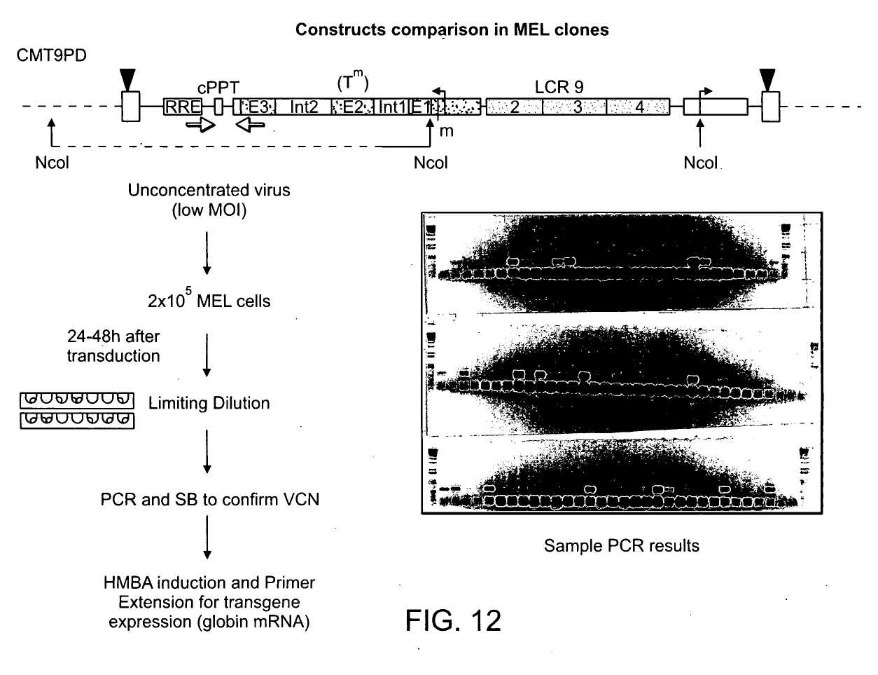

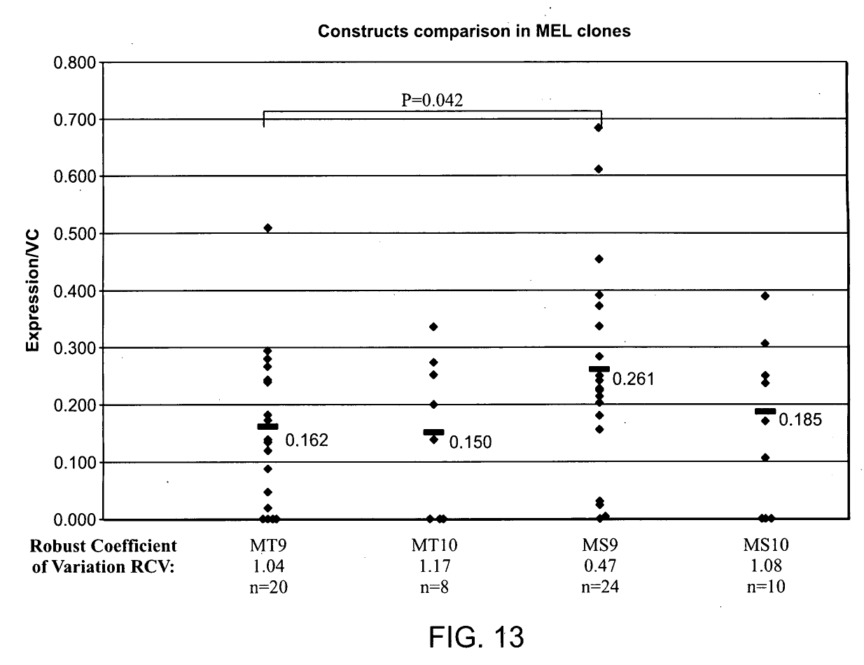

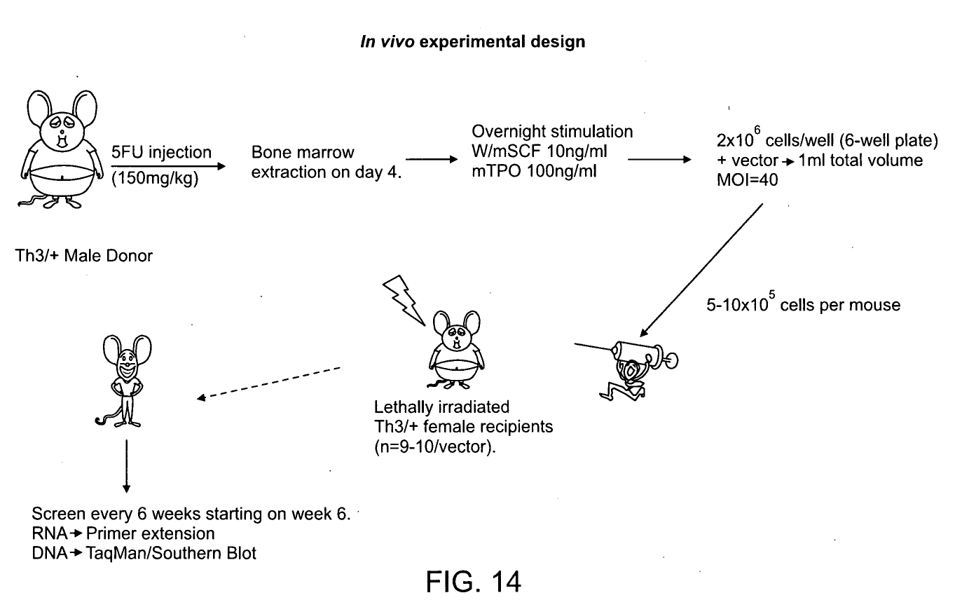

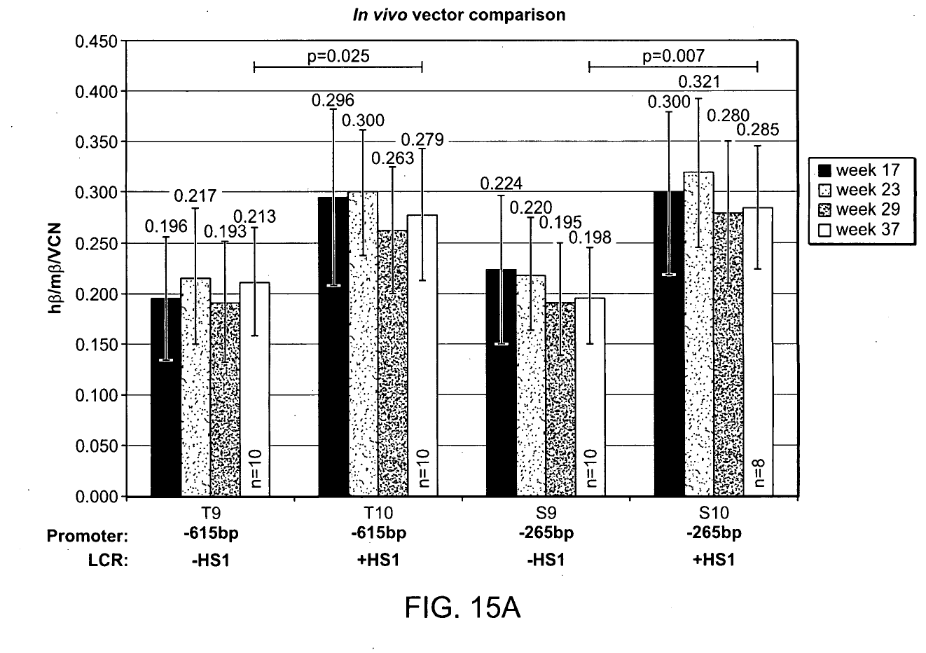

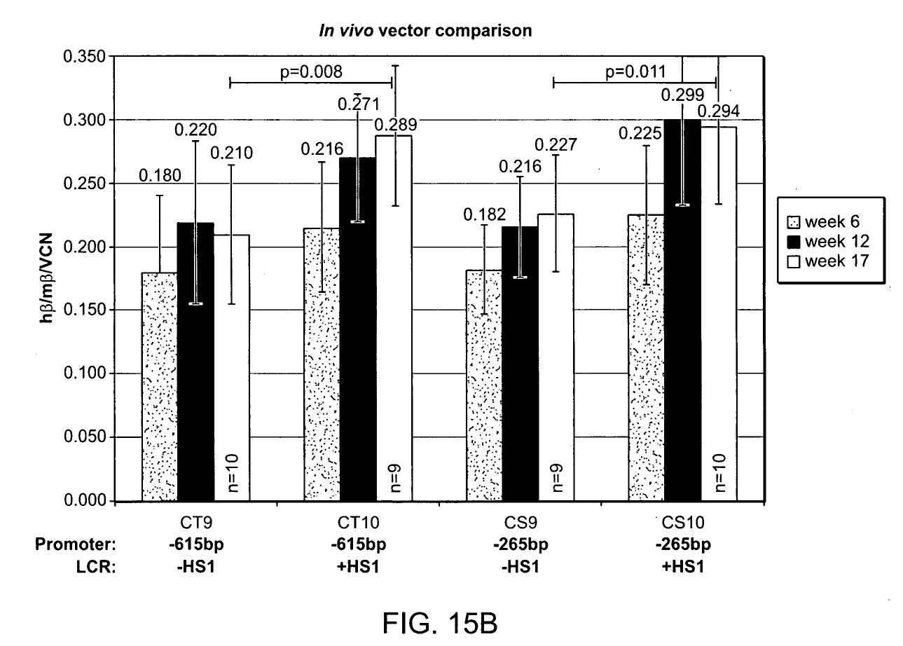

FIGS. 7 and 8 are schematic diagrams showing the structure of the human beta globin gene locus, the TNS9 vector, and the HS1 element of the human β-globin locus control region locus control region (LCR). The TNS9 vector is described by May et al., Nature 406, 82-86 (6 Jul. 2000, which is hereby incorporated by reference in its entirety. The HS1 element, present in T10 and S10, but not T9 and S9, enhances globin expression in primary erythroid cells in vivo. FIG. 9 shows the constructs used to evaluate the HS1 element. FIG. 10 is a schematic diagram that illustrates the strategy used to conduct the experiments described herein. FIG. 11 is a graph showing the results of an in vitro evaluation of HS1. FIG. 12 is a construct comparison in MEL clones. FIG. 13 shows a construct comparison carried out in MEL clones. These experiments indicated that the shorter promoter (−216), which directed higher transgene expression in MEL cells when juxtaposed to HS2, HS3 and HS4, does not do so in vivo. This may reflect differential transcription factor expression in vitro and in vivo or developmentally regulated acquisition of chromatin structure near the transgene. FIG. 14 is a schematic diagram showing in vivo experimental design. FIGS. 15A and 15B are graphs showing an in vivo comparison of various vectors. FIG. 16 compares in vitro and in vivo data.

### Example 8

### Addition of HS1 to Erythroid-Specific Lentiviral Vectors Expressing Human Factor IX Greatly Increase Factor IX Secretion

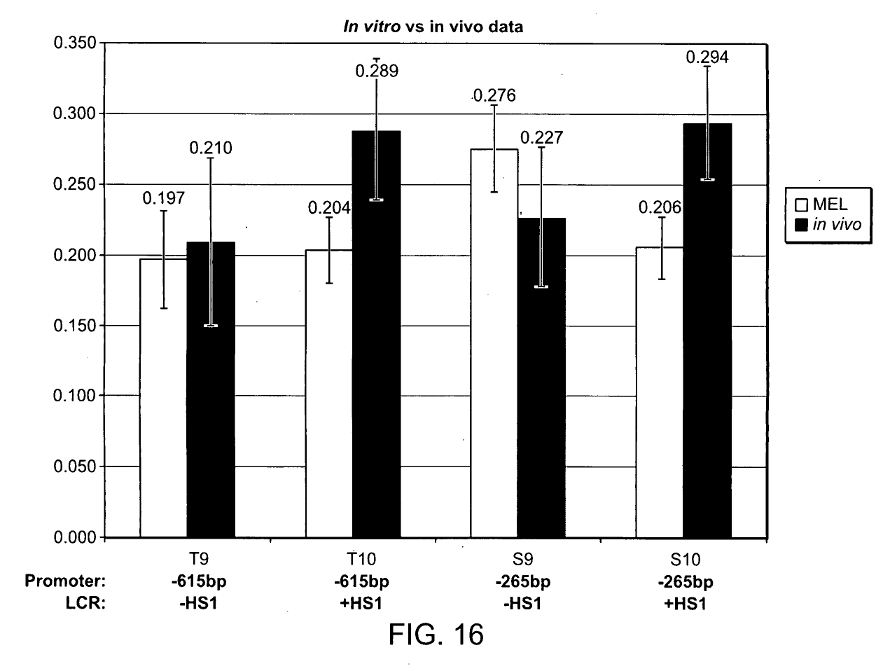

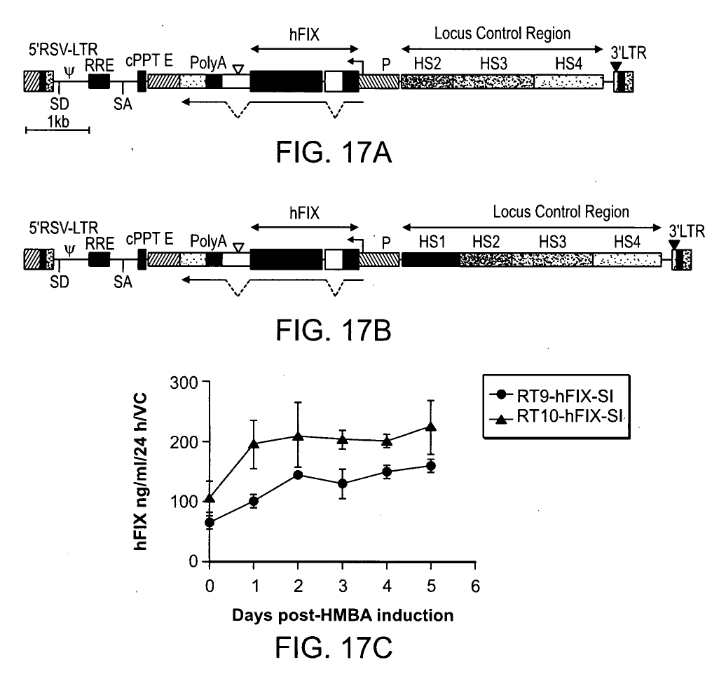

Self-inactivating lentiviral vectors that encode human Factor IX (hFTX) under the transcriptional control of the human β-globin promoter, enhancers and locus control region (LCR), with or without HS1 were constructed. In RT9-hFIX-S1, only HS2-3-4 were used as the classic LCR (FIG. 17A). In RT10-hFIX-S1, HS1 was added to the LCR (FIG. 17B). To investigate the tissue and stage specificity of hFIX expression provided by these two vectors, transduced MEL cell pools were treated with hexamethylene bisacetamide (HMBA) to induce terminal differentiation. Cell culture supernatants were collected every 24 h to determine the hFIX secretion rate as a function of erythroid differentiation. Human FIX expression was measured by ELISA and further normalized to cell number and vector copy number, as determined by Southern blot analysis. Human FIX expression reached a plateau by day 2 after addition of HMBA, which lasted through day 5 (FIG. 17C). Inclusion of the HS1 in the vector increases hFIX expression by approximately 67%, with average outputs of 120 ng/ml and 200 ng/ml per 10⁶ cells, per 24 h, per vector copy (VC, determined by quantitative Southern blot analysis), for RT9-hFIX-SI and RT10-hFIX-SI, respectively. This robust enhancement of expression mediated by HS1 may be promoter length dependent, since the shorter version of human β-globin promoter (265 bp) did not show increased expression of hFIX with the addition of HS1 (data not shown).

In conclusion, these results demonstrate the value of using HS1 to enhance expression of secreted proteins from erythroid cells.

The results described above were carried out using the following methods and materials.

### Vector Construction and Production

T9PD was derived from the previously described TNS9²² by addition of the central polypurine tract (cPPT) element^{74,75} (nucleotides 4781-4898 HIV-1 NCBI Accession number EF36312), and the hPGK-DHFR (human phosphoglycerate kinase promoter driving the dihydrofolate reductase cDNA) cassette⁷⁶, and deletion of the 3′LTR U3 region. The globin transcription unit in T9PD and TNS9.3 (which lacks hPGK-DHFR) is identical to that in TNS9.²² S9 and R9, which encode the shorter 265 bp²⁰ and 130 bp⁷⁷ globin promoters, were derived from T9PD by removal of nucleotides 69933 to 70282 (XbaI to SnaBI site) and nucleotides 69933 to 70417 (NCBI Accession number NG_{—}000007), respectively. V9PD was created by inserting nucleotides from −265 to −1555 amplified from genomic DNA. The HS1 element of LCR was amplified from human genomic DNA (nucleotides 20900-21973 NCBI Accession number NG_{—}000007) (Forward primer: 5′-AAACACCTCTAGGCTATAAGGCAACAGAGC-3′ and Reverse primer 5′-AAGTAAACTTCCACAACCGCAAGC-3′) and kindly provided by Dr. Stefano Rivella. HS1 was inserted in T9PD at the XbaI site lying between the β-globin promoter and HS2. T11PD and T12PD were derived from T10PD and T9PD by truncating the 5′flanking region of HS4 from position 8516 to 9135 (NCBI Accession number NG_{—}000007). The T13PD vector was cloned by removing the 3′ flanking region of HS4 from position 9430 to 9580. The pCL⁷⁸ plasmid encoding the human IFN-β S/MAR (Scaffold/Matrix Attachment Region) was kindly provided by Dr. J. Bode and the 800 bp element inserted into T9PD vector to replace 5′flanking region of HS4, in both orientations (T12SAR1PD and T12SAR2PD). The T12HS3PD construct was obtained by insertion of a 620 bp fragment of the 5′ flanking region of HS3 into T12PD vector in the same orientation. PCR amplified fragments of human glucose-6-phosphate dehydrogenase (G6PD) cDNA were used as spacer sequence.

Viral stocks were generated by triple transfection of the recombinant vectors, pCMVR8.9⁷⁹, and pMD.G⁸⁰ into 293T cells as described.^{22,81} The pseudotyped virions were concentrated by ultracentrifugation and titrated in NIH3T3 cells as described.^{22, 51, 82}

### Vector Copy Number Quantification

For analysis of integrated vector copy number in MEL cells and peripheral blood, genomic DNA was isolated, digested with BamHI, and analyzed by Southern blot²⁰ using a [³²P]dCTP-labelled NcoI-BamHI fragment of the human β-globin gene as a probe. Vector copy number was determined no earlier than 21-days after transduction. To assess integrated vector integrity, Southern blot analysis was performed after NheI digestion, which generates a fragment spanning the entire provirus. Vector copy numbers obtained by Southern blot were corroborated by TaqMan analysis using the following primers and probes:

| | |
|---|---|
| (Gag-specific: | 5′-GGAGCTAGAACGATTCGCAGTT-3′; |
| | 5′-GTTGTAGCTGTCCCAGTATTTGTC-3′; |
| probe: | 5′-ACAGCCTTCTGATGTTTCTAACAGGCCAGG-3′, |
| mouse β-actin specific: | 5′-TCACCCACACTGTGCCCAT-3′; |
| | 5′-AGCCAGGTCCAGACGCAG-3′; |
| probe: | 5′-TACGAGGGCTATGCTCTCCCTCACGC-3′). |

### MEL Cell Transduction and Differentiation

C88 MEL cells were grown in RPMI medium 1640 (GIBCO-Invitrogen, Carlsbad, Calif.) with 10% fetal bovine serum, 100 U/ml penicillin, and 100 ug/ml streptomycin (GIBCO-Invitrogen, Carlsbad, Calif.) in 5% CO₂. Cells were transduced with cell-free vector in the presence of polybrene (4 ug/ml; Sigma). 24 hrs post transduction, cells were selected using methotrexate MTX [5×10⁻⁷M] for 14 days. Cells were induced to differentiation by 5-day culture with 5 mM N,N′-hexamethylene bisacetamide (HMBA, Sigma).⁸³

### Gene Expression Analyses

For β-globin transgene analyses, total RNA was extracted from MEL cells (induced and uninduced) or total mouse peripheral blood using TRIzol reagent (Invitrogen, Carlsbad, Calif.). Quantitative primer extension assays were performed as described previously.²²

### Bone Marrow Chimeras

Animal work was approved by the Institutional Animal Care and Use Committee at MSKCC. Bone marrow was flushed from the femurs and tibias of 8- to 16-week-old male Hbb^{th3/+84} mice 4 days after iv of 5-fluorouracil (5-FU, Pharmacia; 150 mgxkg⁻¹ body weight). Cells were prestimulated for 12 hrs in X-VIVO15 media (BioWhittaker, Cambrex Bio Sci) supplemented with β-mercaptoethanol (0.5 mM) (GIBCO-Invitrogen, Carlsbad, Calif.), L-glutamine (200 mM), penicillin (100 IU/ml) and streptomycin (100 ug/ml), recombinant mouse stem cell factor (rmSCF) (100 ng/ml) and recombinant mouse thrombopoietin (rmTPO) (100 ng/ml) (R&D Systems). Subsequently, cells were transduced in supplemented X-VIVO15 media on RetroNectin-coated 6-well plates (15 ug/ml, TAKARA Shuzo) for 8 hrs. Recipient mice (11- to 18-week old Hbb^{th3/+}mice) were irradiated with 10.5 Gy (split dose 2×5.25 Gy 6 hrs apart) on the day of transplantation. 5×10⁵-1×10⁶ cells were administered by retro-orbital injection.

### Peripheral Blood Analyses

RBC morphology from BM chimeras was assessed in Wright-Giemsa stained PB smears. Red cell counts, white blood cell (WBC) counts, hemoglobin and hematocrits were measured on a Coulter A^{c}T diff. instrument (Beckman Coulter, Brea, Calif.). Reticulocytes were counted after staining with New Methylene Blue N Solution (ACROS Organics, Fairlawn, N.J.). Red cell lysates of freshly collected peripheral blood were analyzed by cellulose acetate electrophoresis (pH 8.5, Helena Laboratories) and quantified as described²² using ImageJ 1.38x software, http://rsb.info.nih.gov/ij/.

### Statistical Analysis

Statistical analysis was done by the Student's t-test using SigmaStat 2.03.0 software (Softek Inc.)
1. Orkin S, Nathan D. Hematology of Infancy and Childhood. Philadelphia, Pa.: WB Saunders Co; 1998: 811-886.
2. Weatherall D. Phenotype-genotype relationships in monogenic disease: lessons from the thalassaemias. Nat. Rev Genet. 2001:2:245-255.
3. Forget B. Molecular mechanism of beta thalassemia. Steinberg M H, Forget B G, Higgs D R, Nagel R L, eds. Disorders of Hemoglobin. Genetics, Pathophysiology and Clinical Management. Cambridge: Cambridge University Press: 2001: 252-276.
4. Kioussis D, Vanin E, deLange T, Flavell R, Grosveld F. Beta-globin gene inactivation by DNA translocation in gamma beta-thalassaemia. Nature. 1983:306:662-666.
5. Curtin P, Pirastu M, Kan Y, Gobert-Jones J, Stephens A, Lehmann H. A distant gene deletion affects beta-globin gene function in an atypical gamma delta beta-thalassemia. J Clin Invest. 1985:76:1554-1558.
6. Driscoll M C, Dobkin C S. Alter B P. gamma delta {beta}—Thalassemia Due to a de novo Mutation Deleting the 5′ {beta}-globin Gene Activation-Region Hypersensitive Sites. PNAS. 1989; 86:7470-7474.
7. Forrester W C, Epner E. Driscoll M C, et al. A deletion of the human beta-globin locus activation region causes a major alteration in chromatin structure and replication across the entire beta-globin locus. Genes Dev. 1990; 4:1637-1649.
8. Stamatoyannopoulos G, Nienhuis A, Majerus P, Varmus H. The Molecular Basis of Blood Disease (ed 3rd). Philadelphia, Pa.: WB Saunders; 1994.
9. Cooley T B, Lee P. A series of cases of splenomegaly in children with anemia and peculiar bone changes. Trans Am Pediatr Soc. 1925:37.
10. Giardina P, Grady R. Chelation therapy in beta-thalassemia: an optimistic update. Semin Hematol. 2001; 38:360-366.
11. Boulad F, Giardina P, Gillio A, et al. Bone Marrow Transplantation for Homozygous {beta}-Thalassemia: The Memorial Sloan-Kettering Cancer Center. Experience. Ann NY Acad. Sci. 1998:850:498-502.
12. Giardini C, Lucarelli G. Bone marrow transplantation in the treatment of thalassemia. Curr Opin Hematol. 1994; 1:170-176.
13. Lucarelli G, Clift R A, Galimberti M, et al. Bone Marrow Transplantation in Adult Thalassemic Patients. Blood. 1999; 93:1164-1167.
14. Tisdale J, Sadelain M. Toward gene therapy for disorders of globin synthesis. Semin Hematol. 2001; 38:382-392.
15. Sadelain M. Genetic treatment of the hnemoglobinopathies: recombinations and new combinations. Br J. Haematol. 1997; 98:247-253.
16. Bank A. Genetic defects in the thalassemias. Curr Top Hematol. 1985:5:1-23.
17. Bank A, Donovan-Peluso M, LaFlamme S, Rund D. Lerner N. Approaches to gene therapy for beta-thalassemia. Birth Defects Orig Artic Ser. 1988:23:339-346.
18. Gale R. Prospects for correction of thalassemia by genetic engineering. Prog Clin Biol Res. 1989:309:141-159.
19. Bank A, Markowitz D, Lerner N. Gene transfer. A potential approach to gene therapy for sickle cell disease. Ann NY Acad. Sci. 1989:565:37-43.
20. Sadelain M, Wang C H J, Antoniou M, Grosveld F, Mulligan R C. Generation of a High-Titer Retroviral Vector Capable of Expressing High Levels of the Human {beta}-Globin Gene. PNAS. 1995; 92:6728-6732.
21. Leboulch P, Huang G, Humphries R, et al. Mutagenesis of retroviral vectors transducing human beta-globin gene and beta-globin locus control region derivatives results in stable transmission of an active transcriptional structure. EMBO J. 1994; 13:3065-3076.
22. May C. Rivella S, Callegari J, et al. Therapeutic haemoglobin synthesis in [beta]-thalassaemic mice expressing lentivirus-encoded human [beta]-globin. Nature. 2000:406:82-86.
23. Puthenveetil G, Scholes J, Carbonell D. et al. Successful correction of the human {beta}-thalassemia major phenotype using a lentiviral vector. Blood. 2004; 104:3445-3453.
24. Plavec I, Papayannopoulou T, Maury C, Meyer F. A human beta-globin gene fused to the human beta-globin locus control region is expressed at high levels in erythroid cells of mice engrafted with retrovirus-transduced hematopoietic stem cells. Blood. 1993; 81:1384-1392.
25. Persons D A, Hargrove P W, Allay E R, Hanawa H, Nienhuis A W. The degree of phenotypic correction of murine beta-thalassemia intermedia following lentiviral-mediated transfer of a human gamma-globin gene is influenced by chromosomal position effects and vector copy number. Blood. 2003:101:2175-2183.
26. Pawliuk R, Westerman K A, Fabry M E, et al. Correction of Sickle Cell Disease in Transgenic Mouse Models by Gene Therapy. Science. 2001; 294:2368-2371.
27. Levasseur D N, Ryan T M, Pawlik K M, Townes T M. Correction of a mouse model of sickle cell disease: lentiviral/antisickling {beta}-globin gene transduction of unmobilized, purified hematopoietic stem cells. Blood. 2003:102:4312-4319.
28. Imren S, Payen E, Westerman K A, et al. Permanent and panerythroid correction of murine beta thalassemia by multiple lentiviral integration in hematopoietic stem cells. PNAS. 2002; 99:14380-14385.
29. Imren S. Fabry M, Westerman K. et al. High-level β-globin expression and preferred intragenic integration after lentiviral transduction of human cord blood stem cells. J Clin Invest. 2004; 114:953-962.
30. Hanawa H, Hargrove P W, Kepes S, Srivastava D K, Nienhuis A W, Persons D A. Extended {beta}-globin locus control region elements promote consistent therapeutic expression of a {gamma}-globin lentiviral vector in murine {beta}-thalassemia. Blood. 2004; 104:2281-2290.
31. Emery D W, Yannaki E, Tubb J, Nishino T, Li Q. Stamatoyannopoulos G. Development of virus vectors for gene therapy of beta chain hemoglobinopathies: flanking with a chromatin insulator reduces gamma-globin gene silencing in vivo. Blood. 2002; 100:2012-2019.
32. Trudel M, Costantini F. A 3′ enhancer contributes to the stage-specific expression of the human beta-globin gene. Genes Dev. 1987; 1:954-961.
33. Antoniou M, deBoer E, Habets G, Grosveld F. The human beta-globin gene contains multiple regulatory regions: identification of one promoter and two downstream enhancers. The EMBO Journal. 1988; 7:377-384.
34. Talbot D, Philipsen S, Fraser P, Grosveld F. Derailed analysis of the site 3 region of the human beta-globin dominant control region. EMBO J. 1990; 9:2169-2177.
35. Philipsen S, Talbot D, Fraser P, Grosveld F. The beta-globin dominant control region: hypersensitive site 2. EMBO J. 1990; 9:2159-2167.
36. Li Q, Harju S, Peterson K R. Locus control regions: coming of age at a decade plus. Trends in Genetics. 1990; 15:403-408.
37. Li Q, Stamatoyannopoulos J A. Position independence and proper developmental control of gamma-globin gene expression require both a 5′ locus control region and a downstream sequence element. Mol Cell Biol. 1994; 14:60S7-6096.
38. Grosveld F, van Assendelft G, Greaves D, Kollias G. Position-independent, high-level expression of the human β-globin gene in transgenic mice. Cell. 1987:51:975-985.
39. Tuan D, Solomon W. Li Q, London I M. The “{beta}-Like-Globin” Gene Domain in Human Erythroid Cells. PNAS. 1985:82:6384-6388.
40. Forrester W C, Thompson C, Elder J T, Groudine M. A Developmentally Stable Chromatin Structure in the Human {beta}-globin Gene Cluster. PNAS. 1986:83:1359-1363.
41. Bulger M, Groudine M. Looping versus linking: toward a model for long-distance gene activation. Genes Dev. 1999; 13:2465-2477.
42. Engel J D. Tanimoto K. Looping, Linking, and Chromatin Activity: New Insights into β-globin Locus Regulation. Cell. 2000; 100:499-502.
43. Blom van Assendelft G, Hanscombe O, Grosveld F, Greaves D. The beta-globin dominant control region activates homologous and heterologous promoters in a tissue-specific manner. Cell. 1989:56:969-977.
44. Ryan T M, Behringer R R, Martin N C, Townes T M, Palmiter R D, Brinster R L. A single erythroid-specific DNase I super-hypersensitive site activates high levels of human beta-globin gene expression in transgenic mice. Genes Dev. 1989; 3:314-323.
45. Forrester W C, Novak U, Gelinas R, Groudine M. Molecular Analysis of the Human {beta}-globin Locus Activation Region. PNAS. 1989; 86:5439-5443.
46. Talbot D, Collis P, Antoniou M, Vidal M, Grosveld F, Greaves D R. A dominant control region from the human [beta]-globin locus conferring integration site-independent gene expression. Nature. 1989; 338:352.
47. Fraser P, Hurst J, Collis P, Grosveld F. DNaseI hypersensitive sites 1, 2 and 3 of the human beta-globin dominant control region direct position-independent expression. Nucl Acids Res. 1990; 18:3503-3508.
48. Sadelain M, Lisowski L, Samakoglu S, Rivella S, May C. Riviere I. Progress Toward the Genetic Treatment of the {beta}-Thalassemias. Ann NY Acad. Sci. 2005:1054:78-91.
49. Sadelain, Rivella, Lisowski, Samakoglu, Riviere. Globin gene transfer for treatment of the?-thalassemias and sickle cell disease. Best Practice & Research Clinical Haematology. 2004; 17:517.
50. Chang A H, Stephan M T, Sadelain M. Stem cell-derived erythroid cells mediate long-term systemic protein delivery. Nat. Biotech. 2006; 24:1017.
51. May C, Rivella S, Chadburn A, Sadelain M. Successful treatment of murine beta-thalassemia intermedia by transfer of the human beta-globin gene. Blood. 2002; 99:1902-1908.
52. Rivella S, May C, Chadburn A, Riviere I, Sadelain M. A novel murine model of Cooley anemia and its rescue by lentiviral-mediated human beta-globin gene transfer. Blood. 2003; 101:2932-2939.
53. Samakoglu S, Lisowski L, Budak-Alpdogan T, et al. A genetic strategy to treat sickle cell anemia by coregulating globin transgene expression and RNA interference. Nat Biotech. 2006; 24:89.
54. Kooren J. Palstra R-J, Klous P. et al. beta-Globin Active Chromatin Hub Formation in Differentiating Erythroid Cells and in p45 NF-E2 Knock-out Mice. J Biol Chem. 2007; 282:16544-16552.
55. Splinter E, Heath H, Kooren J, et al. CTCF mediates long-range chromatin looping and local histone modification in the beta-globin locus. Genes Dev: 2006:20:2349-2354.
56. Tolhuis B, Palstra R, Splinter E, Grosveld F, de Laat W. Looping and Interaction between Hypersensitive. Sites in the Active β-globin Locus. Mol Cell. 2002:10:1453-1465.
57. Wijgerde M, Grosveld F, Fraser P. Transcription complex stability and chromatin dynamics in vivo. Nature. 1995; 377:209.
58. Navas P A, Peterson K R, Li Q, et al. Developmental Specificity of the Interaction between the Locus Control Region and Embryonic or Fetal Globin Genes in Transgenic Mice with an HS3 Core Deletion. Mol Cell Biol. 1998; 18:4188-4196.
59. Navas P A, Peterson K R, Li Q, McArthur M, Stamatoyannopoulos G. The 5′HS4 core element of the human β-globin locus control region is required for high-level globin gene expression in definitive but not in primitive erythropoiesis. Journal of Molecular Biology. 2001; 312:17-26.
60. Kulozik A E, Bail S, Bellan-Koch A, Bartram C R, Kohne E, Kleihaue E. The proximal element of the beta globin locus control region is not functionally required in vivo. J Clin Invest. 1991:87:2142-2146.
61. Berg P E, Williams D M, Qian R-L, et al. A common protein binds to two silencers 5′ to the human {beta}-globin gene, Nucl Acids Res. 1989; 17:8833-8852.
62. Bungert J, Dave U, Lim K C, et al. Synergistic regulation of human beta-globin gene switching by locus control resion elements HS3 and HS4. Genes Dev. 1995; 9:3083-3096.
63. Ellis J, Tan-Un K, Harper A, et al. A dominant chromatin-opening activity in 5′ hypersensitive site 3 of the human beta-globin locus control region. EMBO J. 1996:15:562-568.
64. Collis P, Antoniou M, Grosveld F. Definition of the minimal requirements within the human beta-globin gene and the dominant control region for high level expression. EMBO J. 1990; 9:233-240.
65. Fraser P G, Frank. Locus control regions, chromatin activation and transcription. Current Opinion in Cell Biology. 1998; 10:361-365.
66. Hardison R, Slightom J, Gumucio D, Goodman M, Stojanovic N, Miller W. Locus control regions of mammalian beta-globin gene clusters: combining phylogenetic analyses and experimental results to gain functional insights. Gene. 1997; 205:73-94.
67. Stamatoyannopoulos G, Grosveld F. Hemoglobin switching. In: Stamatoyannopoulos G, Majerus P W, Perlmutter R M, Varmus H, eds. Molecular Basis of Blood Disorders. 3rd ed. Philadelphia, Pa.: WB Saunders: 2001: 135-182.
68. Pasceri P, Pannell D, Wu X, Ellis J. 5′HS1 and the Distal {beta}-Globin Promoter Functionally Interact in Single Copy {beta}-Globin Transgenic Mice. Ann NY Acad Sci. 1998:850:377-381.
69. Pasceri P, Pannell D, Wu X. Ellis J. Full Activity From Human beta-Globin Locus Control Region Transgenes Requires 5′HS1, Distal beta-Globin Promoter, and 3′ beta-Globin Sequences. Blood. 1998; 92:653-663.
70. Milot E, Strouboulis J, Triniborn T, et al. Heterochromatin Effects on the Frequency and Duration of LCR-Mediated Gene Transcription Cell. 1996;S7:105-114.
71. Bender M A, Roach J N, Halow J, et al. Targeted deletion of 5′HS1 and 5′HS4 of the {beta}-globin locus control region reveals additive activity of the DNaseI hypersensitive sites

### Blood. 2001; 98:2022-2027.

72. Fedosyuk H, Peterson K R. Deletion of the human β-globin LCR 5′HS4 or 5′HS1 differentially affects β-like globin gene expression in β-YAC transgenic mice. Blood Cells, Molecules, and Diseases. 2007:39:44-55.
73. Pruzina S, Hanscombe O, Whyatt D, Grosveld F, Philipsen S. Hypersensitive site 4 of the human {beta} globin locus control region. Nucl Acids Res. 1991; 19:1413-1419.
74. Manganini M, Serafini M, Bambacioni F, et al. A Human Immunodeficiency Virus Type 1 po1 Gene-Derived Sequence (cPPT/CTS) Increases the Efficiency of Transduction of Human Nondividing Monocytes and T Lymphocytes by Lentiviral Vectors. Human Gene Therapy. 2002; 13:1793-1807.
75. Sirven A, Pflumio F, Zennou V. et al. The human immunodeficiency virus type-1 central DNA flap is a crucial determinant for lentiviral vector nuclear import and gene transduction of human hematopoietic stem cells. Blood. 2000; 96:4103-4110.
76. Simonsen C C, Levinson A D. Isolation and Expression of an Altered Mouse Dihydrofolate Reductase cDNA. PNAS. 1983; 80:2495-2499.
77. Persons D A, Allay E R, Sawai N, et al. Successful treatment of murine {beta}-thalassemia using in vivo selection of genetically modified, drug-resistant hematopoietic stem cells. Blood. 2003:102:506-513.
78. Schubeler D, Mielke C, Maass K, Bode J. Scaffold/Matrix-Attached Regions Act upon Transcription in a Context-Dependent Manner. Biochemistry. 1996:35:11160-11169.
79. Zufferey R, Nagy D, Mandel R J, Naldini L, Trono D. Multiply attenuated lentiviral vector achieves efficient gene delivery in vivo. Nat Biotech. 1997; 15:871.
80. Ory D S, Neugeboren B A, Mulligan R C. A stable human-derived packaging cell line for production of high titer retrovirus/vesicular stomatitis virus G†pseudotypes. PNAS. 1996; 93:11400-11406.
81. Dull T, Zufferey R, Kelly M, et al. A Third-Generation Lentivirus Vector with a Conditional Packaging System. J Virol. 1998; 72:8463-8471.
82. Gallardo H F, Tan C, Ory D, Sadelain M. Recombinant Retroviruses Pseudotyped With the Vesicular Stomatitis Virus G Glycoprotein Mediate Both Stable Gene Transfer and Pseudotransduction in Human Peripheral Blood Lymphocytes. Blood. 1997; 90:952-957.
83. Nudel U, Salmon J, Fibach E, et al. Accumulation of [alpha]- and [beta]-globin messenger RNAs in mouse erythroleukemia cells. Cell. 1977:12:463.
84. Yang B, Kirby S, Lewis J, Detloff P J, Maeda N, Smithies O. A Mouse Model for {beta}O-Thalassemia. PNAS. 1995; 92:11608-11612.
85. Han X-D. Lin C, Chang J, Sadelain M, Kan Y W. Fetal gene therapy of {alpha}-thalassemia in a mouse model. PNAS. 2007:104:9007-9011.
86. deBoer E, Antoniou M, Mignotte V, Wall L, Grosveld F. The human beta-globin promoter: nuclear protein factors and erythroid specific induction of transcription. The EMBO Journal. 1988; 7:4203-4212.
87. Bode J, Kohwi Y, Dickinson L, et al. Biological significance of unwinding capability of nuclear matrix-associating DNAs. Science. 1992; 255:195-197.
88. Ciavatta D J, Ryan T M, Farmer S C, Townes T M. Mouse Model of Human {beta}0 Thalassemia: Targeted Deletion of the Mouse {beta}maj- and {beta}min-Globin Genes in Embryonic Stem Cells. PNAS. 1995; 92:9259-9263.
89. Vassilopoulos G, Navas P A, Skarpidi E, et al. Correct Function of the Locus Control Region May Require Passage Through a Nonerythroid Cellular Environment. Blood. 1999; 93:703-712.
90. Mpollo M-SEM, Beaudoin M, Berg P E, Beauchemin H, D'Agati V. Trudel M. BP1 is a negative modulator of definitive erythropoiesis. Nucl Acids Res. 2006; 34:5232-5237.
91. Molete J M, Petrykowska H, Bouhassira E E, Feng Y-Q, Miller W, Hardison R C. Sequences Flanking Hypersensitive Sites of the {beta}-Globin Locus Control Region Are Required for Synergistic Enhancement. Mol Cell Biol. 2001; 21:2969-2980.
92. Bungert J, Tanimoto K, Patel S, Liu Q, Fear M, Engel J D. Hypersensitive Site 2†Specifies a Unique Function within the Human beta-Globin Locus Control Region To Stimulate Globin Gene Transcription. Mol Cell Biol. 1999:19:3062-3072.
93. Cunningham J M. Purucker M E, Jane S M, et al. The regulatory element 3′ to the A gamma-globin gene binds to the nuclear matrix and interacts with special A-T-rich binding protein 1 (SATB1), an SAR/MAR-associating region DNA binding protein. Blood. 1994; 84:1298-1308.
94. Dang Q, Amen J, Plavec I. Human Beta Interferon Scaffold Attachment Region Inhibits De Novo Methylation and Confers Long-Term, Copy Number-Dependent Expression to a Retroviral Vector. J Virol. 2000; 74:2671-2678.
95. Auten J, Agarwal M, Chen J, Sutton R Plavec I. Effect of Scaffold Attachment Region on Transgene Expression in Retrovirus Vector-Transduced Primary T Cells and Macrophages. Human Gene Therapy. 1999; 10:1389-1399.
96. Agarwal M, Austin T W, Morel F, Chen J, Bohnlein E, Plavec I. Scaffold Attachment Region-Mediated Enhancement of Retroviral Vector Expression in Primary T Cells. J Virol. 1998:72:3720-3728.
97. Ramezani A, Hawley T S, Hawley R G. Performance- and safety-enhanced lentiviral vectors containing the human interferon-{beta} scaffold attachment region and the chicken {beta}-globin insulator. Blood. 2003:101:4717-4724.
98. Baum C, Dullmann J, Li Z, et al. Side effects of retroviral gene transfer into hematopoietic stem cells. Blood. 2003; 101:2099-2113.
99. Chang A H, Sadelain M. The Genetic Engineering of Hematopoietic Stem Cells: the Rise of Lentiviral Vectors, the Conundrum of the LTR and the Promise of Lineage-restricted Vectors. Mol Ther. 2007.15:445.
100. Aker M, Tubb J, Groth A C, et al. Extended Core Sequences from the cHS4 Insulator Are Necessary for Protecting Retroviral Vectors from Silencing Position Effects. Human Gene Therapy. 2007; 18:333-343.
101. Arumugam P I, Scholes J, Perelman N, Xia P, Yee J-K, Malik P. Improved Human [beta]-globin Expression from Self-inactivating Lentiviral Vectors Carrying the Chicken Hypersensitive Site-4 (cHS4) Insulator Element. Mol Ther. 2007.
102. Emery D W, Yannaki E, Tubb J, Stamatoyannopoulos G. A chromatin insulator protects retrovirus vectors from chromosomal position effects. PNAS. 2000:97:9150-9155.
103. Rivella S, Callegari J A, May C, Tan C W, Sadelain M. The cHS4 Insulator Increases the Probability of Retroviral Expression at Random Chromosomal Integration Sites. J Virol. 2000; 74:4679-4687.
104. Sadelain M, Boulad F, Galanello R, et al. Therapeutic options for patients with severe beta-thalassemia: the need for globin gene therapy. Human Gene Therapy. 2007:18:1-9.
105. Myers R M, Tilly K, Maniatis T. Fine Structure Genetic Analysis of a Beta-Globin Promoter. Science. 1986; 232:613-618.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

The contents of all references (including literature references, issued patents, published patent applications, and co-pending patent applications) cited throughout this application are hereby expressly incorporated herein in their entireties by reference.

What is claimed is:

1. A recombinant vector comprising:
(a) a nucleotide sequence encoding a heterologous protein;
(b) a nucleotide sequence encoding an enhancer of a β-globin locus control region (LCR) which consists of operably joined Dnase1 hypersensitive site (HS) spanning-fragments; and
(c) a nucleotide sequence encoding an HS1 fragment consisting of about nucleotides 20900 to 21973 of the human β-globin LCR sequence of GenBank Accession No. NG_{—}000007.3 (NG7) or the corresponding sequences from a mammalian β-globin LCR;
said vector providing expression of said protein when introduced into a mammal in vivo.

2. The recombinant vector of claim 1, wherein the vector further comprises a β-globin promoter.

3. The recombinant vector of claim 1, wherein the HS spanning-fragments span HS2, HS3 and HS4.

4. The recombinant vector of claim 1, wherein the HS spanning-fragments span HS2 and HS3.

5. The recombinant vector of claim 1, wherein the HS spanning-fragments consist essentially of an HS2-spanning nucleotide fragment extending between BstXI and SnaBI restriction sites of said LCR, an HS3-spanning nucleotide fragment extending between BamHI and HindIII restriction sites of said LCR and an HS4-spanning nucleotide fragment extending between BamHI and BanII restriction sites of said LCR.

6. The recombinant vector of claim 1, wherein the HS2 spanning-fragment extending between BstXI and SnaBI restriction sites of said LCR consists of about nucleotides 16241 to 17093 of the sequence of GenBank Accession No. NG_{—}000007.3 (NG7).

7. The recombinant vector of claim 1, wherein the HS3 spanning-fragment extending between BamHI and HindIII restriction sites of said LCR consists of about nucleotides 12066 to 13360 of the sequence of GenBank Accession No. NG_{—}000007.3 (NG7).

8. The recombinant vector of claim 1, wherein the HS4 spanning-fragment extending between BamHI and BanII restriction sites of said LCR consists of about nucleotides 8497 to 9576 of the sequence of GenBank Accession No. NG_{—}000007.3 (NG7).

9. The recombinant vector of claim 1, wherein the heterologous protein is a globin or Factor IX.

10. The recombinant vector of claim 9, wherein the globin is a β-globin.

11. The recombinant vector of claim 9, wherein the globin is a γ-globin.

12. The recombinant vector of claim 9, wherein the globin is a α-globin.

13. The recombinant vector of claim 10, wherein the β-globin is a human β-globin.

14. The recombinant vector of claim 9, wherein the globin is a mutant globin.

15. The recombinant vector of claim 9, wherein the globin is a wild-type globin.

16. The recombinant vector of claim 1, wherein the vector is a lentiviral vector.

17. The recombinant vector of claim 1, wherein the vector is HIV-1-derived.

18. The recombinant vector of claim 1, wherein the vector is selected from the group consisting of T9, T10, S9, S10, T12, and V9.

19. The recombinant vector of claim 2, wherein the HS1 fragment lies between the β-globin promoter and an HS-spanning fragment, and wherein the HS-spanning fragment is an HS2-spanning fragment.

20. The recombinant vector of claim 2, wherein the β-globin promoter is ≧ about 265 bp in length.

21. The recombinant vector of claim 2, wherein the β-globin promoter is about 615 bp in length.

22. The recombinant vector of claim 9, wherein the globin is encoded by a nucleotide sequence that has at least 1 intron comprising interfering RNA.

23. The recombinant vector of claim 22, wherein the interfering RNA is selected from the group consisting of an antisense RNA, a short hairpin RNA, an siRNA and a microRNA.

24. The recombinant vector of claim 23, wherein the interfering RNA selectively degrades a messenger RNA transcript of an undesired host cell gene.

25. The recombinant vector of claim 24, wherein the undesired host cell gene contains a mutation.

26. The recombinant vector of claim 1, further comprising a nucleotide sequence encoding a dihydrofolate reductase.

27. The recombinant vector of claim 26, wherein the dihydrofolate reductase is a human dihydrofolate reductase.

28. The recombinant vector of claim 1, further comprising a central polypurine tract (cPPT) element, a human phosphoglycerate kinase promoter driving a dihydrofolate reductase cDNA (hPGK-DHFR) cassette, or a deletion of the 3′ U3 LTR region, or any combination thereof.

29. The recombinant vector of claim 1, wherein the HS-spanning fragments are HS3 and HS4-spanning fragments and 2 GATA-1 binding sites are present at the junction between the HS3 and HS4-spanning fragments.

30. The recombinant vector of claim 9, which provides expression of greater than two copies of globin per cell when introduced into a mammal in vivo.

31. The recombinant vector of claim 1, wherein the heterologous protein is selected from the group consisting of a clotting factor, enzyme, hormone, growth factor, anti-angiogenic factor, antibody and antigen.

32. A method of treating a disorder in a subject comprising administering to a subject in need thereof a therapeutically effective amount of the recombinant vector of claim 1, thereby treating the disorder in the subject.

33. The method of claim 32, wherein the vector is a lentivector that is used to transduce a hematopoietic progenitor or stem cell.

34. The method of claim 33, wherein the hematopoietic progenitor or stem cell is transduced in vitro or in vivo.

35. The method of claim 33, wherein the lentivector has a selectable marker, and a selection step using an anti-folate is additionally performed.

36. The method of claim 32, wherein the disorder is a hemoglobinopathy.

37. The method of claim 36, wherein the hemoglobinopathy is a hemoglobinopathy selected from the group consisting of hemoglobin C disease, hemoglobin sickle cell disease (SCD), sickle cell anemia, hereditary anemia, thalassemia, β thalassemia, thalassemia major, thalassemia intermedia, α-thalassemia, and hemoglobin H disease.

38. The method of claim 37, wherein the disorder is a factor IX deficiency.

39. The method of claim 37, wherein the vector is administered to hematopoietic stem cells of the subject.

40. The method of claim 32, wherein the subject is a mammal.

41. The method of claim 40, wherein the subject is human.

42. A pharmaceutical composition comprising the recombinant vector of claim 1 and a pharmaceutically acceptable carrier or excipient.

43. A packaged pharmaceutical comprising the recombinant vector of claim 1 and associated instructions for using said vector to treat a disorder in a subject.

44. A method of producing a heterologous protein, the method comprising transfecting a cell with the recombinant vector of claim 1, and expressing the heterologous protein in the cell, thereby producing the heterologous protein.

45. The method of claim 44, wherein the cell is a hematopoietic progenitor or stem cell.

46. The method of claim 44, further comprising the step of isolating the protein from the cell.
